SEQUENCE LISTING (14 sequences)

SEQ ID NO: 1 - protein, 7 aa, Homo sapiens
SRATMPG

SEQ ID NO: 2 - protein, 14 aa, Homo sapiens
SLGQRSQKLAAALE

SEQ ID NO: 3 - protein, 8 aa, Homo sapiens
YNSKRQPN

SEQ ID NO: 4 - protein, 8 aa, Homo sapiens
ENKTRKIM

SEQ ID NO: 5 - protein, 10 aa, Homo sapiens
RDRATAIQPG

SEQ ID NO: 6 - protein, 5 aa, Homo sapiens
SSKSP

SEQ ID NO: 7 - protein, 1606 aa, Homo sapiens
MVVFINAKIKSLIKLFKKKTVSNLDEESRWTVHYTAPWHQQENVFLPTTRPPCVEDLHRQAKLNLKSVLRECDKLRHDGYRSSQYYSQGPTFAANASPFCDDYQDEDEETDQKCSLSSSEEERFISIRRPKTPASSDFSDLNTQTNWTKSLPLPTPEEKMRQQAQTVQADVVPINITGENFDRQASLRRSLIYTDTLVRRPKKVKRRKTITGVPDNIQKELASGTGQDDADGHSVYTPDHYSTLGRFNSCRSAGQRSETRDSSCQTEDVKVVPPSMRRIRAQKGQGIAAQMGHFSGSSGNMSVLSDSAGIVFPSRLDSDAGFHSLPRSGARANIQSLEPRLGALGPAGDMNGTFLYQRGHPQADENLGHLGGASGTGTLLRPKSQELRHFESENIMSPACVVSPHATYSTSIIPNATLSSSSEVIAIPTAQSAGQRESKSSGSSHARIKSRDHLISRHAVKGDPQSPGRHWNEGHATILSQDLDPHSPGEPALLSLCDSAVPLNAPANRENGSQAMPYNCRNNLAFPAHPQDVDGKSESSYSGGGGHSSSEPWEYKSSGNGRASPLKPHLATPGYSTPTSNMSSCSLDQTSNKEDAGSLYSEDHDGYCASVHTDSGHGSGNLCNSSDGFGNPRHSVINVFVGRAQKNQGDRSNYQDKSLSRNISLKKAKKPPLPPSRTDSLRRIPKKSSQCNGQVLNESLIATLQHSLQLSLPGKSGSSPSQSPCSDLEEPWLPRSRSQSTVSAGSSMTSATTPNVYSLCGATPSQSDTSSVKSEYTDPWGYYIDYTGMQEDPGNPAGGCSTSSGVPTGNGPVRHVQEGSRATMPQVPGGSVKPKIMSPEKSHRVISPSSGYSSQSNTPTALTPVPVFLKSVSPANGKGKPKPKVPERKSSLISSVSISSSSTSLSSSTSTEGSGTMKKLDPAVGSPPAPPPPPVPSPPFPCPADRSPFLPPPPPVTDCSQGSPLPHSPVFPPPPPEALIPFCSPPDWCLSPPRPALSPILPDSPVSLPLPPPLLPSSEPPPAPPLDPKFMKDTRPPFTNSGQPESSRGSLRPPSTKEETSRPPMPLITTEALQMVQLRPVRKNSGAEAAQLSERTAQEQRTPVAPQYHLKPSAFLKSRNSTNEMESESQPASVTSSLPTPAKSSSQGDHGSAAERGGPVSRSPGAPSAGEAEARPSPSTTPLPDSSPSRKPPPISKKPKLFLVVPPPQKDFAVEPAENVSEALRAVPSPTTGEEGSVHSREAKESSAAQAGSHATHPGTSVLEGGAAGSMSPSRVEANVPMVQPDVSPAPKQEEPAENSADTGGDGESCLSQQDGAAGVPETNAAGSSSEACDFLKEDGNDEVMTPSRPRTTEDLFAAIHRSKRKVLGRRDSDDDHSRNHSPSPPVTPTGAAPSLASPKQVGSIQRSIRKSSTSSDNFKALLLKKGSRSDTSARMSAAEMLKNTDPRFQRSRSEPSPDAPESPSSCSPSKNRRAQEEWAKNEGLMPRSLSFSGPRYGRSRTPPSAASSRYSMRNRIQSSPMTVISEGEGEAVEPVDSIARGALGAAEGCSLDGLAREEMDEGGLLCGEGPAASLQPQAPGPVDGTASAEGREPSPQCGGSLSEES

SEQ ID NO: 8 - protein, 69 aa, Homo sapiens
AMVPCDNVSSCPSSDTCCQLTSGEWGCCPIPEAVCCSDHQHCCPQGYTCVAEGQCQKLAAALEHHHHHH

SEQ ID NO: 9 - protein, 487 aa, Homo sapiens [sequence omitted]

SEQ ID NO: 10 - protein, 882 aa, Homo sapiens
MKYSCCALVLAVLGTELLGSLCSTVRSPRFRGRIQQERKNIRPNIILVLTDDQDVELGSLQVMNKTRKIMEHGGATFINAFVTTPMCCPSRSSMLTGKYVHNHNVYTNNENCSSPSWQAMHEPRTFAVYLNNTGYRTAFFGKYLNEYNGSYIPPGWREWLGLIKNSRFYNYTVCRNGIKEKHGFDYAKDYFTDLITNESINYFKMSKRMYPHRPVMMVISHAAPHGPEDSAPQFSKLYPNASQHITPSYNYAPNMDKHWIMQYTGPMLPIHMEFTNILQRKRLQTLMSVDDSVERLYNMLVETGELENTYIIYTADHGYHIGQFGLVKGKSMPYDFDIRVPFFIRGPSVEPGSIVPQIVLNIDLAPTILDIAGLDTPPDVDGKSVLKLLDPEKPGNRFRTNKKAKIWRDTFLVERGKFLRKKEESSKNIQQSNHLPKYERVKELCQQARYQTACEQPGQKWQCIEDTSGKLRIHKCKGPSDLLTVRQSTRNLYARGFHDKDKECSCRESGYRASRSQRKSQRQFLRNQGTPKYKPRFVHTRQTRSLSVEFEGEIYDINLEEEEELQVLQPRNIAKRHDEGHKGPRDLQASSGGNRGRMLADSSNAVGPPTTVRVTHKCFILPNDSIHCERELYQSARAWKDHKAYIDKEIEALQDKIKNLREVRGHLKRRKPEECSCSKQSYYNKEKGVKKQEKLKSHLHPFKEAAQEVDSKLQLFKENNRRRKKERKEKRRQRKGEECSLPGLTCFTHDNNHWQTAPFWNLGSFCACTSSNNNTYWCLRTVNETHNFLFCEFATGFLEYFDMNTDPYQLTNTVHTVERGILNQLHVQLMELRSCQGYKQCNPRPKNLDVGNKDGGSYDRTVMGWMGRLISPVSLQTSTGKA

SEQ ID NO: 11 - protein, 1141 aa, Homo sapiens
MDDSGELGGLETMETLTELGDELTLGDIDEMLQFVSNQVGEFPDLFSEQLCSSFPGSGGSGSSSGSSGSSSSSSNGRGSSSGAVDPSVQRSFTQVTLPSFSPSAASPQAPTLQVKVSPTSVPTTPRATPILQPRPQPQPQPQTQLQQQTVMITPTFSTTPQTRIIQQPLIYQNAATSFQVLQPQVQSLVTSSQVQPVTIQQQVQTVQAQRVLTQTANGTLQTLAPATVQTVAAPQVQQVPVLVQPQIIKTDSLVLTTLKTDGSPVMAAVQNPALTALTTPIQTAALQVPTLVGSSGTILTTMPVMMGQEKVPIKQVPGGVKQLEPPKEGERRTTHNIIEKRYRSSINDKIIELKDLVMGTDAKMHKSGVLRKAIDYIKYLQQVNHKLRQENMVLKLANQKNKLLKGIDLGSLVDNEVDLKIEDFNQNVLLMSPPASDSGSQAGFSPYSIDSEPGSPLLDDAKVKDEPDSPPVALGMVDRSRILLCVLTFLCLSFNPLTSLLQWGGAHDSDQHPHSGSGRSVLSFESGSGGWFDWMMPTLLLWLVNGVIVLSVFVKLLVHGEPVIRPHSRSSVTFWRHRKQADLDLARGDFAAAAGNLQTCLAVLGRALPTSRLDLACSLSWNVIRYSLQKLRLVRWLLKKVFQCRRATPATEAGFEDEAKTSARDAALAYHRLHQLHITGKLPAGSACSDVHMALCAVNLAECAEEKIPPSTLVEIHLTAAMGLKTRCGGKLGFLASYFLSRAQSLCGPEHSAVPDSLRWLCHPLGQKFFMERSWSVKSAAKESLYCAQRNPADPIAQVHQAFCKNLLERAIESLVKPQAKKKAGDQEEESCEFSSALEYLKLLHSFVDSVGVMSPPLSRSSVLKSALGPDIICRWWTSAITVAISWLQGDDAAVRSHFTKVERIPKALEVTESPLVKAIFHACRAMHASLPGKADGQQSSFCHCERASGHLWSSLNVSGATSDPALNHVVQLLTCDLLLSLRTALWQKQASASQAVGETYHASGAELAGFQRDLGSLRRLAHSFRPAYRKVFLHEATVRLMAGASPTRTHQLLEHSLRRRTTQSTKHGEVDAWPGQRERATAILLACRHLPLSFLSSPGQRAVLLAEAARTLEKVGDRRSCNDCQQMIVKLGGGTAIAAS

SEQ ID NO: 12 - protein, 976 aa, Homo sapiens
MAQVAMSTLPVEDEESSESRMVVTFLMSALESMCKELAKSKAEVACIAVYETDVFVVGTERGRAFVNTRKDFQKDFVKYCVEEEEKAAEMHKMKSTTQANRMSVDAVEIETLRKTVEDYFCFCYGKALGKSTVVPVPYEKMLRDQSAVVVQGLPEGVAFKHPENYDLATLKWILENKAGISFIIKRPFLEPKKHVGGRVMVTDADRSILSPGGSCGPIKVKTEPTEDSGISLEMAAVTVKEESEDPDYYQYNIQGPSETDDVDEKQPLSKPLQGSHHSSEGNEGTEMEVPAEDDDYSPPSKRPKANELPQPPVPEPANAGKRKVREFNFEKWNARITDLRKQVEELFERKYAQAIKAKGPVTIPYPLFQSHVEDLYVEGLPEGIPFRRPSTYGIPRLERILLAKERIRFVIKKHELLNSTREDLQLDKPASGVKEEWYARITKLRKMVDQLFCKKFAEALGSTEAKAVPYQKFEAHPNDLYVEGLPENIPFRSPSWYGIPRLEKIIQVGNRIKFVIKRPELLTHSTTEVTQPRTNTPVKEDWNVRITKLRKQVEEIFNLKFAQALGLTEAVKVPYPVFESNPEFLYVEGLPEGIPFRSPTWFGIPRLERIVRGSNKIKFVVKKPELVISYLPPGMASKINTKALQSPKRPRSPGSNSKVPEIEVTVEGPNNNNPQTSAVRTPTQTNGSNVPFKPRGREFSFEAWNAKITDLKQKVENLFNEKCGEALGLKQAVKVPFALFESFPEDFYVEGLPEGVPFRRPSTFGIPRLEKILRNKAKIKFIIKKPEMFETAIKESTSSKSPPRKINSSPNVNTTASGVEDLNIIQVTIPDDDNERLSKVEKARQLREQVNDLFSRKFGEAIGMGFPVKVPYRKITINPGCVVVDGMPPGVSFKAPSYLEISSMRRILDSAEFIKFTVIRPFPGLVINNQLVDQSESEGPVIQESAEPSQLEVPATEEIKETDGSSQIKQEPDPTW

SEQ ID NO: 13 - DNA, 21 bp, Artificial Sequence
  Synthetic Construct
tgctaaggacaacgttatcgg

SEQ ID NO: 14 - DNA, 18 bp, Artificial Sequence
  Synthetic Construct
ttgataccggacgttcac

(19) United States
(12) Patent Application Publication
(10) Pub. No.: US 2013/0072400 A1
(43) Pub. Date: Mar. 21, 2013

(54) METHOD FOR THE DIAGNOSIS/PROGNOSIS OF COLORECTAL CANCER

(71) Applicants: José Ignacio Casal Álvarez, Madrid (ES); Rodrigo Barderas Manchado, Madrid (ES); Ingrid Henriette Suzanne Babel, Madrid (ES)

(72) Inventors: José Ignacio Casal Álvarez, Madrid (ES); Rodrigo Barderas Manchado, Madrid (ES); Ingrid Henriette Suzanne Babel, Madrid (ES)

(73) Assignee: CONSEJO SUPERIOR DE INVESTIGACIONES CIENTIFICAS, Madrid (ES)

(21) Appl. No.: 13/697,749

(22) PCT Filed: May 13, 2011
(86) PCT No.: PCT/ES2011/070346

§ 371 Date: Dec. 5, 2012

(30) Foreign Application Priority Data
May 13, 2010 (ES) P201030708

(51) Int. Cl. G01N 33/574; C12N 9/12; C12N 9/14; C07K 7/08; C40B 40/02; C40B 40/10; C07K 7/06; C12N 7/00; C40B 30/04

(52) U.S. Cl. 506/9; 436/501; 435/7.1; 435/5; 435/235.1; 435/194; 435/195; 435/7.92; 506/14; 506/18; 530/328; 530/327; 530/330; 530/329

(57) ABSTRACT

The present invention relates to a method for obtaining useful data for the diagnosis, prognosis or monitoring of colorectal cancer (CRC) progression, to a method for the diagnosis of CRC, to a method for the prognosis of CRC and to a kit for carrying out said methods.

### FIELD OF THE INVENTION

The present invention is comprised within the field of biomedicine. It specifically relates to a method for obtaining useful data for the diagnosis, prognosis or monitoring of colorectal cancer (CRC) progression, to a method for the diagnosis of CRC, to a method for the prognosis of CRC and to a kit for carrying out said methods.

### BACKGROUND OF THE INVENTION

Colorectal cancer (CRC) is the second most prevalent cancer in the Western world. The disease develops over decades and involves multiple genetic events. Despite the fact that CRC is one of the best characterized solid tumors from a genetic viewpoint, it continues to be one of the main causes of death in developed countries because of the late diagnosis of patients due to the waiting time that passes to perform certain diagnostic tests, such as colonoscopy.

Today there are few proteins that have been described as effective biomarkers of CRC (carcinoembryonic antigen (CEA), CA19.9 and CA125) (Crawford et al. 2003. Journal of surgical oncology 84 (4), 239-248; Duffy et al. 2007 Eur J Cancer 43 (9), 1348-1360) and they are not specific enough to perform clinical screenings with a view to detect CRC (Locker et al. 25 2006. J Clin Oncol 24 (33), 5313-5327).

Proteomic analyses are being actively used for identifying new biomarkers. In different earlier proteomic studies, differentially expressed proteins in CRC tissue have been identified by means of using antibody microarrays and 2D-DIGE, including isoforms and post-transductional modifications responsible for modifications in signaling pathways (Alfonso et al. 2005. Proteomics 5(10), 2602-2611; Kopf et al. 2005. Proteomics 5(9), 2412-2416; Madoz-Gurpide et al. 2007. Mol Cell Proteomics 6 (12), 2150-2164; Alfonso et al. 2008. Journal of Proteome Research 7 (10), 4247-4255). These two approaches have allowed identifying a broad collection of potential tumor markers of CRC tissue which are currently under research.

However, the implementation of non-invasive and simpler diagnostic methods which allow early detection of CRC must be based on identifying proteins or antibodies detectable in serum or plasma (Hanash et al. 2008. Nature 452 (7187), 571-579; Hudson et al. 2007. Proceedings of the National Academy of Sciences of the United States of America 104 (44), 17494-17499). The existence of an immune response to cancer in humans has been shown by the presence of autoantibodies in serum from cancer patients. Different human proteins (autoantigens) can thereby be affected before or during the formation of the tumor, being able to produce an immune response once released (Hudson et al. 2007. Proceedings of the National Academy of Sciences of the United States of America 104 (44), 17494-17499; Wang et al. 2005. The New England Journal of Medicine 353 (12), 1224-1235; Sreekumar et al. 2004. J Natl Cancer Inst 96 (11), 834-843). Said autoantibodies can be detected in early stages of the disease and even before the cancer can be detected by means of other techniques, indicating their enormous potential as biomarkers of the disease. These tumor proteins can be affected by point mutations, have anomalous folding, be overexpressed, aberrantly glycosylate, be truncated or undergo aberrant degradation as is the case of p53, HER2, NY-ES01 or MUC1, respectively (Chen et al. 1997. Proceedings of the National Academy of Sciences of the United States of America 94 (5), 1914-1918; Schubert et al. 2000. Nature 404 (6779), 770-774; Ulanet et al. 2003. Proceedings of the National Academy of Sciences of the United States of America 100 (21), 12361-12366). In fact, tumor-associated autoantigens (TAAs) have previously been characterized in CRC using different approaches (Scanlan et al. 1998. International Journal of Cancer 76 (5), 652-658). Several authors have described some TAA panels as biomarkers of CRC, among which STK4/MST1 protein is found (Tan et al. 2009. Journal 276: 6880-6904; Babel et al. 2009. Molecular and Cellular Proteomics 8: 2382-2395; WO 2010/136629).

Nevertheless, the diagnostic validity of the autoantibodies associated with CRC identified until now still requires an independent validation for their generalized use in the diagnosis/prognosis of CRC.

Therefore, there is a need for biomarkers which allow the diagnosis of CRC, its classification in the different stages of tumor progression, the prognosis of disease progression, the evaluation of its response to a specific treatment and the detection of the recurrence or the spread of CRC, by means of a simple, effective and non-invasive method.

### BRIEF DESCRIPTION OF THE INVENTION

The present invention relates to a method for obtaining useful data for the diagnosis, prognosis or monitoring of colorectal cancer (CRC) progression, to a method for the diagnosis of CRC, to a method for the prognosis of CRC and to a kit for carrying out said methods.

The present invention therefore provides a response to the need for biomarkers which allow the diagnosis of CRC, its classification in the different stages of tumor progression, the prognosis of disease progression, the evaluation of its response to a specific treatment and the detection of the recurrence or the spread of CRC, by means of a simple, effective and non-invasive method.

Blood is usually the optimal biological fluid based on non-invasive methods for massive screening of large populations of patients for diagnostic purposes. On one hand, serum and plasma are easy to obtain, and on the other hand, blood circulation facilitates the contact of the blood with all the tissues of the human body, including contact with tumor tissue and its representative antigens in the case of cancer patients. The release of these tumor associated antigens probably occurs at a very low concentration in plasma and probably experience proteolysis in a short time period. In contrast, antibodies are very stable molecules which have been used for years in different clinical immunoassays, which facilitates standardizing assays. The use of autoantibodies is also beneficial because the immune system amplifies the response, facilitating identification and quantification.

Phage microarrays (occasionally identified in this description as “phages-peptides”) have been used in the present invention to identify autoantibodies present in serum from CRC patients at different stages.

Six phages containing sequences homologous to NHSL1, GRN, MST1, SULF1, SREBF2 and GTF2i proteins were selected. The combination of MST1 and SULF1 recombinant proteins with the 4 other phage sequences allowed predicting the disease with 72% sensitivity and 87% specificity, with an Area Under the Curve (AUC) of 0.83. If the age of the patient is further taken into account, the AUC is 0.91. These markers further allow grouping the results by discriminating not only the sick individuals but also the different stages of the disease. The detection of this panel of autoantibodies in serum is therefore a simple and non-invasive method for the diagnosis/prognosis of CRC.

The present invention provides new biomarkers for the diagnosis, the prognosis, the tracking of CRC and its diagnosis from a blood sample from a subject in which the autoantibodies against an antibody capturing entity (ACE) are detected and/or quantified, wherein said ACE is selected from the group consisting of:
(i) an ACE comprising the amino acid sequence shown in SEQ ID NO: 4 or a variant thereof containing an epitope recognizable by an autoantibody;
(ii) an ACE comprising the amino acid sequence shown in SEQ ID NO: 2 or a variant thereof containing an epitope recognizable by an autoantibody;
(iii) an ACE comprising the amino acid sequence shown in SEQ ID NO: 6 or a variant thereof containing an epitope recognizable by an autoantibody;
(iv) an ACE comprising the amino acid sequence shown in SEQ ID NO: 3 or a variant thereof containing an epitope recognizable by an autoantibody, wherein said ACE is not MST1 protein;
(v) an ACE comprising the amino acid sequence shown in SEQ ID NO: 5 or a variant thereof containing an epitope recognizable by an autoantibody;
(vi) an ACE comprising the amino acid sequence shown in SEQ ID NO: 1 or a variant thereof containing an epitope recognizable by an autoantibody; and
(vii) any combination of said ACEs (i)-(vi).

Generally, the greater the number of biomarkers used, the more precise the diagnosis/prognosis of CRC.

The present invention therefore provides a specific autoantibody signature of CRC consisting of the presence of new disease specific biomarkers, with potential for diagnosing CRC using sera from patients with greater specificity and sensitivity than the biomarkers described until now.

The present invention further provides a method for the diagnosis of CRC based on determining SULF1 protein levels.

The inventive aspects of the present invention are described in the attached claims.

### BRIEF DESCRIPTION OF THE DRAWINGS

FIG. 1 shows the response of the autoantibodies to the six specific phages. Intensity of the signal of each phage with the CRC sera and the control sera. The results show the data after normalization and on a scale of arbitrary units (a.u.).

FIG. 2 shows the competitive analysis between the phage peptides and their respective homologous proteins. A. An ELISA competitive assay was conducted between the phages displaying peptides with homology to SULF1 and MST1 and the respective recombinant proteins. GST was used as a negative inhibition control. Increasing amounts of the recombinant proteins were pre-incubated with the serum from patients and their respective phage binding was tested by means of ELISA (Vertical bars: light gray, recombinant protein; dark gray, GST). EBNA1 protein was used as a control to demonstrate that inhibition was specific of the protein used as inhibitor and that no error was introduced in the assay (data not shown). B. Schematic location of the peptides with homology to SULF1 and MST1 within the recombinant proteins. The position of the peptide in the protein is highlighted in the figure. The vertical lines correspond to potential phosphorylation sites. The different amino acids between the phage and protein sequence are in lower case letters.

FIG. 3 shows the analysis of SULF1, MST1, GTF2i, NHSL1, GRN and SREBF2 expression in cell lines and tissue from CRC patients. A. The gene expression levels of the proteins the peptide of which is displayed in T7 phages was determined by means of meta-analysis using the Oncomine database. The p values are indicated in the figure. Relative gene expression levels were found for NHSL1, SREBF2, GTF2i, SULF1, MST1 and GRN. B. Immunodetection in SULF1 and MST1 membrane in colorectal cancer cell lines compared with control cell lines and paired tissues of CRC belonging to stages I, II and III. An anti-tubulin antibody was used as a loading control. C, Data of the tissue microarray for GTF2i and GRN obtained from the Human Protein Atlas WebPage.

FIG. 4 shows the predictive values of MST1 and SULF1 proteins. A. Mean absorbance values obtained with CRC sera and the controls by means of indirect ELISA. The dots represent the individual value for each serum. The error bars represent the standard deviation value. The images of the polyacrylamide gels correspond to the recombinant proteins used in the ELISA assays. B. Both proteins were capable of discriminating control sera from sera from CRC patients with p values <0.0001 and 0.0006 for MST1 and SULF1, respectively. The AUC for MST1 was 0.75 (95% CI=0.647-0.829) with sensitivity and specificity of 60.0% and 82.6%, respectively, using 0.63 as a cutoff point. The AUC was 0.72 (95% CI=0.617-0.805) for SULF1, with sensitivity and specificity of 68% and 67.4%, respectively, using 0.36 as a cutoff point. C and D, ROC curves for CEA and the combination of 4 phages, 2 proteins and the age variable, resulting in AUC values of 0.81 and 0.89, respectively.

FIG. 5 shows the survival analysis using autoantibodies against MST1 and NHSL1 proteins. The Kaplan-Meier survival curves were calculated using an independent set of 95 sera from CRC patients to analyze the effect of the presence of autoantibodies on absolute survival of CRC patients.

FIG. 6 shows the validation of the combination of four phages with MST1 and SULF1 proteins in the diagnosis of colorectal cancer. Behavior of the combination of MST1 and NHSLlphages GTF2i, NHSL1, GRN and SREBF2 and MST1 and NHSL1 proteins in the validation test. A. Behavior of CRC samples versus healthy controls. B. Behavior of CRC samples versus reference sera. C. Behavior of healthy sera versus tumor sera. D. Dotplot showing the individual probability of being classified as a CRC patient for each of the subjects with different pathologies. Most of the samples were classified below the probability value of 0.5 (dotted vertical line).

### DETAILED DESCRIPTION OF THE INVENTION

### Definitions

The meaning of some terms and expressions as they are used in the present description are indicated below to aid in understanding.

As it is used herein, the term “antibody” refers to immunoglobulin molecules and immunologically active portions of immunoglobulin molecules, i.e., molecules containing an antigen fixing site binding specifically (immunoreacting) with an antigen, such as a protein for example. There are 5 isotypes or main classes of immunoglobulins: immunoglobulin M (IgM), immunoglobulin D (IgD), immunoglobulin G (IgG), immunoglobulin A (IgA) and immunoglobulin E (IgE).

As it is used herein, the term “autoantibody” applies to an antibody reacting against an antigen present in the subject's organism, even if the reaction occurs only in vitro, and whether or not it causes pathological effects in vivo.

As it is used herein, the term “colorectal cancer” or “CRC”, also called colon cancer, includes any type of neoplasias of the colon, rectum and appendix, as well as any histological subtype typically occurring in colon cancer, e.g., transitional carcinoma cells, squamous carcinoma and adenocarcinoma cells, any clinical subtype, e.g., surface, invasive muscle or metastatic disease cancer, or any TNM stage including T0-T4, N0-N2 and M0-M1 tumors. Patients can be classified in different groups with respect to the stage of the tumor. The classification of colon cancer is an estimate of the penetration of a particular cancer. It is carried out for investigational purposes, diagnostic purposes and for determining the best method of treatment. The system for the classification of colorectal cancer depends on the extent of local invasion, on the degree of lymphatic nodes involved and on if distant metastasis exists. The most common classification system is the TNM (for tumors/nodes/metastasis) system, of the American Joint Committee on Cancer (AJCC). The TNM system assigns a number based on three categories. “T” indicates the degree of invasion of the intestinal wall, “N” the degree of involvement of lymphatic nodes and “M” the degree of metastasis. The broadest stage of cancer is usually mentioned as a number I, II, III, IV derived from the TNM value grouped by the prognosis, a higher number indicates a more advanced cancer and a worse prognosis. Details of the classification are indicated in Table 1.

TABLE 1
TNM system for the classification of CRC

| AJCC Stage | TNM Stage | Criteria of TNM stages for CRC |
|---|---|---|
| Stage 0 | Tis N0 M0 | Tis: The tumor confined to the |
| | | mucosa; cancer-in-situ |
| Stage I | T1 N0 M0 | T1: The tumor invades the mucosa |
| Stage I | T2 N0 M0 | T2: The tumor invades the actual |
| | | muscles |
| Stage II-A | T3 N0 M0 | T3: The tumor invades the |
| | | subserosal layer or beyond |
| | | (other organs not involved) |
| Stage II-B | T4 N0 M0 | T4: The tumor invades adjacent |
| | | organs or perforates the |
| | | visceral peritoneum |
| Stage III-A | T1-2 N1 M0 | N1: Metastasis of 1 to 3 |
| | | regional lymphatic nodes. T1 or |
| | | T2. |
| Stage III-B | T3-4 N1 M0 | N1: Metastasis of 1 to 3 |
| | | regional lymphatic nodes. T3 or |
| | | T4. |
| Stage III-C | any T, N2 M0 | N2: Metastasis of 4 or more |
| | | regional lymphatic nodes. Any T. |
| Stage IV | any T, any N, | M1: Presence of distant |
| | M1 | metastasis. Any T, any N. |

As it is used herein, the term “antibody capturing entity” (ACE) refers to a macromolecular entity binding specifically to an antibody (or autoantibody). In a particular embodiment, said ACE comprises a peptide or a protein binding specifically to an antibody (or autoantibody). Said peptide can either be immobilized on a support or exposed on the phage surface. In a preferred particular embodiment, said ACE is a peptide, a protein or a phage on the surface of which said peptide or said protein is exposed. If desired, said ACE can be immobilized on a solid support.

As it is used herein, the term “sample” refers but is not limited to a tissues and/or biological fluids from a subject, obtained by means of any method known by a person skilled in the art which serves for carrying out any of the methods provided by the present invention; i.e., said biological sample must be a sample susceptible to containing antibodies, e.g., autoantibodies against SULF1, MST1 proteins, etc., or against ACEs comprising the amino acid sequences shown in SEQ ID NO: 1-6, or variants thereof containing epitopes recognizable by autoantibodies, etc. By way of non-limiting illustration, said biological sample can be a blood, urine, saliva, serum, or plasma sample, a buccal or buccal-pharyngeal swab, a surgical specimen, a specimen obtained from a biopsy or autopsy, etc. In a particular embodiment, said sample is a biological fluid. In a preferred embodiment for the detection of autoantibodies, the sample from the subject is blood, plasma or blood serum. In another particular embodiment, said sample is a tissue sample. In a preferred embodiment for the quantification of SULF1 protein level, said sample is preferably a colorectal tissue sample or tumor tissue sample, etc., obtained by conventional methods, for example, by means of a biopsy, resection, etc.

As it is used herein, the term “SULF1 protein” includes SULF1 protein and variants thereof; in a particular embodiment, said protein is the protein with NCBI database accession number (May 1, 2011 version) EAW86954.1 and its amino acid sequence is SEQ ID NO: 10.

As it is used herein, the term “NHSL1 protein” includes NHSL1 protein and variants thereof; in a particular embodiment, said protein is the protein with NCBI database accession number (May 1, 2011 version) NP_{—}001137532.1 and its amino acid sequence is SEQ ID NO: 7.

As it is used herein, the term “GRN protein” includes GRN protein and variants thereof; in a particular embodiment, said protein is the protein with NCBI database accession number (May 1, 2011 version) 2JYT and its amino acid sequence is SEQ ID NO: 8.

As it is used herein, the term “MST1 protein” includes MST1 protein and variants thereof; in a particular embodiment, said protein is the protein with NCBI database accession number (May 1, 2011 version) AAA83254.1 and its amino acid sequence is SEQ ID NO: 9.

As it is used herein, the term “SREBF2 protein” includes SREBF2 protein and variants thereof; in a particular embodiment, said protein is the protein with NCBI database accession number (May 1, 2011 version) NP_{—}004590.2 and its amino acid sequence is SEQ ID NO: 11.

As it is used herein, the term “GTF2i protein” includes GTF2i protein and variants thereof; in a particular embodiment, said protein is the protein with NCBI database accession number (May 1, 2011 version) NP_{—}001157108.1 and its amino acid sequence is SEQ ID NO: 12.

As it is used herein, the term “variant” refers to a protein or peptide substantially homologous to another protein or peptide, for example, to the peptides the amino acid sequences of which are shown in SEQ ID NO:1 to 6, to SULF1, MST1, NHSL1, GRN, SREBF2 or GTF2i proteins, etc. A variant generally includes additions, deletions or substitutions of one or more amino acids. The person skilled in the art will understand that the amino acid sequences referred to in this description can be chemically modified, for example, by means of physiologically relevant chemical modifications, such as phosphorylations, acetylations, glycosylations or methylations. According to the present invention, said variants are recognized by autoantibodies against the protein or peptide in question. Variants of said peptides or proteins include peptides or proteins showing at least 25%, at least 40%, at least 60%, at least 70%, at least 80%, at least 90%, at least 95%, at least 96%, at least 97%, at least 98% or at least 99% sequence identity with respect to certain amino acid sequences of peptides or proteins. The degree of identity between two amino acid sequences can be determined by conventional methods, for example, by means of standard sequence alignment algorithms known in the state of the art, such as BLAST for example (Altschul S. F. et al. Basic local alignment search tool. J Mol. Biol. 1990; 215(3):403-10).

Method for Detecting Autoantibodies in Subjects with CRC

In a first aspect, the invention relates to a method for detecting an autoantibody in a subject suspected of having colorectal cancer (CRC), hereinafter first method of the invention, comprising:
a) contacting a sample from said subject with an antibody capturing entity (ACE), wherein said ACE is selected from the group consisting of:
   (i) an ACE comprising the amino acid sequence shown in SEQ ID NO: 4 or a variant thereof containing an epitope recognizable by an autoantibody;
   (ii) an ACE comprising the amino acid sequence shown in SEQ ID NO: 2 or a variant thereof containing an epitope recognizable by an autoantibody;
   (iii) an ACE comprising the amino acid sequence shown in SEQ ID NO: 6 or a variant thereof containing an epitope recognizable by an autoantibody;
   (iv) an ACE comprising the amino acid sequence shown in SEQ ID NO: 3 or a variant thereof containing an epitope recognizable by an autoantibody, wherein said ACE is not MST1 protein;
   (v) an ACE comprising the amino acid sequence shown in SEQ ID NO: 5 or a variant thereof containing an epitope recognizable by an autoantibody;
   (vi) an ACE comprising the amino acid sequence shown in SEQ ID NO: 1 or a variant thereof containing an epitope recognizable by an autoantibody; and
   (vii) any combination of said ACEs (i)-(vi); and
b) detecting the formation of an autoantibody-ACE complex,
   wherein the detection of said autoantibody-ACE complex is indicative of the presence of said autoantibody in said subject.

The sample will generally be a biological sample susceptible to containing antibodies from a subject, and it can be obtained by conventional methods known by those of average skill in the art, depending on the nature of the sample. In a particular embodiment, said biological sample is a blood, serum or plasma sample which can be obtained by any conventional method, for example, by means of a blood extraction, etc. Blood is usually the optimal biological fluid to be used in non-invasive methods for massive screening of large populations of subjects for diagnostic purposes. On one hand, serum and plasma are easy to obtain, and on the other hand, blood circulation facilitates the contact of the blood with all the tissues of the human body, including contact with tumor tissue and its representative antigens in the case of cancer patients.

The first method of the invention comprises contacting a sample from a subject suspected of having CRC with an ACE selected from ACEs (i) to (vi), indicated in Table 2, and their combinations [step a)], under conditions allowing the formation of an autoantibody-ACE complex.

TABLE 2
ACEs

| | |
|---|---|
| (i) | ACE comprising the amino acid sequence shown |
| | in SEQ ID NO: 4 or a variant thereof |
| | containing an epitope recognizable by an |
| | autoantibody. |
| (ii) | ACE comprising the amino acid sequence shown |
| | in SEQ ID NO: 2 or a variant thereof |
| | containing an epitope recognizable by an |
| | autoantibody. |
| (iii) | ACE comprising the amino acid sequence shown |
| | in SEQ ID NO: 6 or a variant thereof |
| | containing an epitope recognizable by an |
| | autoantibody. |
| (iv) | ACE comprising the amino acid sequence shown |
| | in SEQ ID NO: 3 or a variant thereof |
| | containing an epitope recognizable by an |
| | autoantibody; wherein said ACE is not MST1 |
| | protein. |
| (v) | ACE comprising the amino acid sequence shown |
| | in SEQ ID NO: 5 or a variant thereof |
| | containing an epitope recognizable by an |
| | autoantibody. |
| (vi) | ACE comprising the amino acid sequence shown |
| | in SEQ ID NO: 1 or a variant thereof |
| | containing an epitope recognizable by an |
| | autoantibody. |

The suitable conditions for the formation of the autoantibody-ACE complex to take place are known by the persons skilled in the art. If the sample contains autoantibodies against said ACEs, then the corresponding autoantibody-ACE complex will be formed; otherwise, said complex will not be formed.

Although said ACEs could be together in the same medium, in practice it is advantageous for said ACEs to be separated from one another. The ACEs can be in solution or suspension in a suitable medium, or can alternatively be deposited or supported on a support [e.g., a microtiter plate, beads (magnetic or non-magnetic), columns, matrices, membranes, etc.] These materials can be used in the suitable forms, such as films, sheets, plates, etc., or they can be used to coat inert carriers (e.g., paper, glass, plastic films, etc.). In a particular embodiment, the sample to be analyzed is contacted with said ACEs, separated from one another, and deposited on a suitable support.

The detection of said autoantibodies against the ACEs mentioned can be carried out by conventional methods known by those of average skill in the art. In a particular embodiment, the detection of said autoantibodies is carried out by means of immunoassay; illustrative, non-limiting examples of immunoassays known in the state of the art include immunoblot, Enzyme-linked Immunosorbent Assay (ELISA), linear immunoassay (LIA), radioimmunoassay (RIA), immunofluorescence (IF), immunohistochemistry (IHC), protein microarrays, phage microarrays, etc. The person skilled in the art will also understand that other methods based for example on electrophoretic or chromatographic techniques can be used for detecting said autoantibodies.

In a particular embodiment, the detection of autoantibodies against one or more ACEs is done by means of an ELISA. The ELISA technique is based on the premise that an immunoreagent (e.g., an antigen or an antibody) is immobilized on a solid support, and then that system is contacted with a fluid phase containing the complementary reagent which can be bound to a marker compound. There are different types of ELISA, for example, direct ELISA, indirect ELISA or sandwich ELISA.

The detection of autoantibodies against one or more ACE/ACEs by means of ELISA, for example by means of indirect ELISA, generally comprises the following steps: (a) covering a solid support with one or more ACEs, preferably separated from one another; (b) incubating the covered support of step (a) with a sample, such as a biological sample from the subject to be studied, under conditions allowing the formation of an autoantibody-ACE complex; and (c) adding a secondary antibody, which recognizes the autoantibody against the ACE/ACEs, conjugated or bound to a marker compound.

In another particular embodiment, the detection of autoantibodies against one or more ACEs is done by means of a protein microarray. A protein microarray consists of a collection of proteins immobilized on a solid support in a regular and pre-established arrangement. There are several important factors to be taken into account in the design of protein microarrays, among which, for example, the nature of the support on which the proteins (or suitable fragments thereof) are immobilized, the protein immobilization technique, the format of the microarray, the capturing agent used or the method of detection to be used are found. Different formats, supports and techniques which can be used for carrying out this inventive aspect are known in the state of the art.

The detection of autoantibodies against one or more ACEs by means of a protein microarray generally comprises the following steps: (a) covering a solid support with said ACE/ACEs, preferably separated from one another; (b) incubating the covered support of step (a) with a sample, such as a biological sample from the subject to be studied, under conditions allowing the formation of an immunocomplex of the autoantibody against the ACE/ACEs present in said sample with the corresponding antigenic determinants present in said ACEs; and (c) adding a secondary antibody, which recognizes the autoantibody against the ACE/ACEs, conjugated or bound to a marker compound.

In another particular embodiment, the detection of autoantibodies against one or more ACEs is done by means of a phage microarray. A phage microarray consists of a collection of peptides exposed on the surface of phages. In a particular embodiment, said peptides are fused to T7 phage capsid protein 10B. Said phages are immobilized on a solid support in a regular and pre-established arrangement. There are several important factors to be taken into account in the design of phage microarrays such as, for example, the nature of the support on which the phages are immobilized, the immobilization technique, the format of the microarray or the method of detection to be used. Different formats, supports and techniques that can be used for carrying out this preferred aspect of the method of the invention are known in the state of the art.

The detection of autoantibodies against one or more ACE/ACEs by means of a phage microarray generally comprises the following steps: (a) covering a solid support with a phage lysate, for example, a T7 phage lysate, having a peptide or an amino acid sequence susceptible to being recognized by an autoantibody exposed on the phage surface; (b) incubating the covered support of step (a) with a sample, such as a biological sample from the subject to be studied, under conditions allowing the formation of an autoantibody-ACE complex; and (c) adding a secondary antibody, which recognizes the autoantibody against the ACE/ACEs, conjugated or bound to a marker compound. In a specific embodiment, said phage microarray comprises a phage selected from the group consisting of:
(i) a phage comprising the amino acid sequence shown in SEQ ID NO: 4 or a variant thereof containing an epitope recognizable by an autoantibody, wherein said amino acid sequence is exposed on the phage surface;
(ii) a phage comprising the amino acid sequence shown in SEQ ID NO: 2 or a variant thereof containing an epitope recognizable by an autoantibody, wherein said amino acid sequence is exposed on the phage surface;
(iii) a phage comprising the amino acid sequence shown in SEQ ID NO: 6 or a variant thereof containing an epitope recognizable by an autoantibody, wherein said amino acid sequence is exposed on the phage surface;
(iv) a phage comprising the amino acid sequence shown in SEQ ID NO: 3 or a variant thereof containing an epitope recognizable by an autoantibody, wherein said amino acid sequence is exposed on the phage surface;
(v) a phage comprising the amino acid sequence shown in SEQ ID NO: 5 or a variant thereof containing an epitope recognizable by an autoantibody, wherein said amino acid sequence is exposed on the phage surface;
(vi) a phage comprising the amino acid sequence shown in SEQ ID NO: 1 or a variant thereof containing an epitope recognizable by an autoantibody, wherein said amino acid sequence is exposed on the phage surface; and
(vii) any of the combinations of (i) to (vi).

The marker bound to the secondary antibody referred to in these techniques is a compound capable of giving rise to a chromogenic, fluorogenic, radioactive and/or chemiluminescent signal which allows the detection, identification and, optionally, quantification of the amount of the autoantibody versus the ACE/ACEs present in the analyzed sample. In a particular embodiment, said marker compound is selected from the group consisting of radioisotopes, enzymes, fluorophores or any molecule susceptible to being conjugated with another molecule or detected and/or quantified directly. This marker compound can bind to the autoantibody directly, or through another compound. Illustrative non-limiting examples of said marker compounds binding directly to the autoantibody include enzymes, such as alkaline phosphatase, peroxidase, etc., radioactive isotopes, such as ³²P, ³⁵S, etc., fluorochromes, such as fluorescein, etc., or metal particles, for their direct detection by means of colorimetry, auto-radiography, fluorometry, or metallography, respectively.

The detection of the autoantibodies can be carried out by applying a single technique or it can be carried out by applying a combination of two or more techniques; by way of illustration, some autoantibodies can be detected by means of an ELISA and others by means of a protein microarray, or some by means of an ELISA and others by means of a phage microarray, or some by means of a protein microarray and others by means of a phage microarray, etc.

In a particular embodiment, the sample to be analyzed is contacted with a single ACE selected from the group of ACEs (i)-(vi) shown in Table 2, and their combinations, under conditions allowing the formation of an autoantibody-ACE complex for the purpose of identifying autoantibodies against said ACE. In another particular embodiment, said biological sample is contacted with two or more of said ACEs susceptible to being recognized by said autoantibodies, separated from one another, optionally deposited on a suitable support, for the purpose of identifying autoantibodies against said ACEs.

In a particular embodiment, the first method of the invention comprises the detection of an autoantibody against an ACE comprising the amino acid sequence shown in SEQ ID NO: 4 or a variant thereof containing an epitope recognizable by an autoantibody, and furthermore the detection of an autoantibody selected from the group consisting of: (i) an autoantibody against an ACE comprising the amino acid sequence shown in SEQ ID NO: 2 or a variant thereof containing an epitope recognizable by an autoantibody; (ii) an autoantibody against an ACE comprising the amino acid sequence shown in SEQ ID NO: 6 or a variant thereof containing an epitope recognizable by an autoantibody; (iii) an autoantibody against an ACE comprising the amino acid sequence shown in SEQ ID NO: 3 or a variant thereof containing an epitope recognizable by an autoantibody; (iv) an autoantibody against an ACE comprising the amino acid sequence shown in SEQ ID NO: 5 or a variant thereof containing an epitope recognizable by an autoantibody; (v) an autoantibody against an ACE comprising the amino acid sequence shown in SEQ ID NO: 1 or a variant thereof containing an epitope recognizable by an autoantibody; and (vi) any combination of autoantibodies (i) to (v). In a more specific form of said particular embodiment, said first method of the invention comprises the detection of an autoantibody against an ACE comprising the amino acid sequence shown in SEQ ID NO: 4 or a variant thereof containing an epitope recognizable by an autoantibody, and furthermore the detection of any 1, 2, 3, 4, or 5 of said autoantibodies (i) to (v) previously indicated.

In a specific embodiment, said ACE is SULF1 protein or a variant or fragment thereof containing an epitope recognizable by an autoantibody, whereas in another specific embodiment, said ACE is a phage comprising the amino acid sequence shown in SEQ ID NO: 4 or a variant thereof containing an epitope recognizable by an autoantibody, wherein said amino acid sequence is exposed on the phage surface.

In another particular embodiment, the first method of the invention comprises the detection of an autoantibody against an ACE comprising the amino acid sequence shown in SEQ ID NO: 1 or a variant thereof containing an epitope recognizable by an autoantibody, and furthermore the detection of an autoantibody selected from the group consisting of: (i′) an autoantibody against an ACE comprising the amino acid sequence shown in SEQ ID NO: 4 or a variant thereof containing an epitope recognizable by an autoantibody; (ii′) an autoantibody against an ACE comprising the amino acid sequence shown in SEQ ID NO: 2 or a variant thereof containing an epitope recognizable by an autoantibody; (iii′) an autoantibody against an ACE comprising the amino acid sequence shown in SEQ ID NO: 6 or a variant thereof containing an epitope recognizable by an autoantibody; (iv′) an autoantibody against an ACE comprising the amino acid sequence shown in SEQ ID NO: 3 or a variant thereof containing an epitope recognizable by an autoantibody; (v′) an autoantibody against an ACE comprising the amino acid sequence shown in SEQ ID NO: 5 or a variant thereof containing an epitope recognizable by an autoantibody; and (vi′) any combination of autoantibodies (i′) to (v′). In a more specific form, said first method of the invention comprises the detection of an autoantibody against an ACE comprising the amino acid sequence shown in SEQ ID NO: 1 or a variant thereof containing an epitope recognizable by an autoantibody, and furthermore the detection of any 1, 2, 3, 4, or 5 of said autoantibodies (i′) to (v′) previously indicated.

Step b) of the method for detecting autoantibodies of the invention comprises detecting the formation of an autoantibody-ACE complex. This step can be carried out by conventional methods known by those of average skill in the art, for the detection of the formation of antibody-antigen complexes (in this case, autoantibody-ACE).

In a particular embodiment, by way of non-limiting illustration, for the detection of said complex, a conjugate comprising an antibody recognizing the autoantibody and a marker (labeled secondary antibody) can be added under conditions allowing the formation of an (autoantibody-ACE)-antibody/marker complex and detecting the formation of said complex. If the biological sample contains autoantibodies against one or more of said ACEs, then the autoantibody-ACE complex will have been previously formed, whereby when said complex is contacted with said conjugate comprising the antibody and the marker in suitable conditions, (autoantibody-ACE)-antibody/marker complex is formed, which will be viewed by means of the suitable technique depending on the marker used, as mentioned below; whereas, otherwise, i.e., when the biological sample does not contain autoantibodies against said ACE/ACEs then said (autoantibody-ACE)-antibody/marker complex will not be formed. The suitable conditions for the formation of this latter complex to take place are known by the persons skilled in the art.

Virtually any indicator reagent which allows detecting said (autoantibody-ACE)-antibody/marker complex can be used in putting the present invention into practice. By way of non-limiting illustration, said marker can be an enzyme catalyzing a detectable reaction (e.g., peroxidase, glycosidase, alkaline phosphatase, glucose-6-phosphate dehydrogenase, β-galactosidase, β-glucosidase, β-glucuronidase, etc.), a compound generating a signal when it forms part of said complex (e.g., a fluorescent compound or fluorophore, such as fluorescein, rhodamine, etc.; a (chemi)luminescent compound, such as a dioxetane, an acridinium, a phenanthridinium, ruthenium, luminol, etc.), a radioactive element (e.g., sulfur, iodine, etc.), etc. In a particular embodiment, said marker is a peroxidase. The selection of a particular marker is not critical, provided that it is capable of producing a signal by itself or together with one or more additional substances. The (autoantibody-ACE)-antibody/marker complex formed can thus be detected or displayed by any suitable technique, depending on the chosen marker, known by those of average skill in the art, using the suitable devices, for example, by means of techniques based on colorimetric, fluorometric, (chemi)luminescent, radioactive methods, etc., all of them known by those of average skill in the art.

The conjugate comprising said antibody which recognizes said autoantibody and said marker can be obtained by conventional methods known by those of average skill in the art.

By way of illustration, when the marker is an enzyme, the detection of the complex in question can be carried out by contacting said complex with a suitable substrate and, optionally, with suitable enzymatic amplification agents and/or activators. Illustrative non-limiting examples of said substrates include:

For alkaline phosphatase:
Chromogenic: substrates based on p-nitrophenyl phosphate (p-NPP), 5-bromo-4-chloro-3-indolyl phosphate/nitroblue tetrazolium (BCIP/NPT), etc. Fluorogenic: 4-methylumbeliphenyl phosphate (4-MUP), 2-(5″-chloro-2′-phosphoryloxyphenyl)-6-chloro-4-(3H)-quinazolinone (CPPCQ), 3,6-fluorescein-diphosphate (3,6-FDP), etc.

For peroxidases:
Chromogenic: substrates based on 2,2-azinobis(3-ethylbenzothiazoline-6-sulfonic) (ABTS) acid, o-phenylenediamine (OPT), 3,3′,5,5′-tetramethylbenzidine (TMB), o-dianisidine, 5-aminosalicylic acid, 3-dimethylaminobenzoic (DMAB) acid and 3-methyl-2-benzothiazolinehydrazone (MBTH), 3-amino-9-ethylcarbazole (AEC) and 3,3′-diaminobenzidine (DAB) tetrachloride, etc.
Fluorogenic: 4-hydroxy-3-methoxyphenylacetic acid, reduced phenoxazines and reduced benzothiazines, including the reagent Amplex® Red, Amplex UltraRed, reduced dihydroxanthenes, etc.

For glycosidases:
Chromogenic: substrates based on o-nitrophenyl-β-D-galactoside (o-NPG), p-nitrophenyl-β-D-galactoside and 4-methylumbeliphenyl-β-D-galactoside (MUG) for β-D-galactosidase, etc.
Fluorogenic: resorufin β-D-galactopyranoside, fluorescein digalactoside (FDG), fluorescein diglucuronide, 4-methylumbelliferyl beta-D-galactopyranoside, carboxyumbelliferyl beta-D-galactopyranoside, fluorinated coumarin beta-D-galactopyranosides, etc.

Therefore, by means of putting the first method of the invention into practice, it is possible to detect and obtain autoantibodies against the ACEs indicated in Table 2. Additionally, the level or amount of said autoantibodies against said ACEs present in the sample under study could be determined (quantified) if desired because the signal generated by some markers (e.g., enzymes, etc.) is proportional to the amount of autoantibody present in said sample.

Optionally, if desired, the autoantibody-ACE complex can be isolated by means of conventional techniques, for example, by means of using immunoprecipitation techniques, etc., and the sequence of the autoantibody binding to the ACE can be subsequently sequenced by means of conventional proteomic methods described in the art, such as the determination of the peptide fingerprint or MS/MS analysis (Vikas Dhingraa, et al. 2005. International Journal of Pharmaceutics 299 (1-2):1-18; Hanash S M et al. Nature. 2008 Apr. 3; 452(7187):571-9).

According to the first method of the invention, the detection of the autoantibody-ACE complex is indicative of the presence of the corresponding specific autoantibody (or autoantibodies) against said ACE/ACEs in the analyzed sample and, therefore, in the analyzed subject.

In a particular embodiment, the formation of said autoantibody-ACE complex in said sample can be correlated with a diagnosis of CRC in the subject the analyzed sample is from, or with the prognosis of said disease, or with tracking said disease progression. In the sense used in this description, the term “correlate” refers to comparing the presence or amount of the indicator in a subject (e.g., a subject suspected of having CRC) with its presence or amount in subjects having said disease (CRC), or predisposed to develop it, or in subjects free of said disease.

As it is used herein, the term “diagnosis” generally refers to the process whereby a disease, nosological entity, syndrome, or any disease-health condition is identified. Particularly, the term “diagnosis of colorectal cancer (or CRC)” refers to the capacity to identify or detect the presence of CRC; this detection, as it is understood by a person skilled in the art, does not claim to be correct in 100% of the analyzed samples. However, it requires that a statistically significant amount of the analyzed samples are classified correctly. The amount that is statistically significant can be established by a person skilled in the art by using different statistical tools; illustrative, non-limiting examples of said statistical tools include determining confidence intervals, determining the p-value, the Student's t-test or Fisher's discriminant functions, etc. (see, for example, Dowdy and Wearden, Statistics for Research, John Wiley & Sons, New York 1983). The confidence intervals are preferably at least 90%, at least 95%, at least 97%, at least 98% or at least 99%. The p-value is preferably less than 0.1, less than 0.05, less than 0.01, less than 0.005 or less than 0.0001. The teachings of the present invention advantageously allow correctly detecting CRC in at least 50%, preferably in at least 60%, more preferably in at least 70%, even more preferably in at least 80%, or still even more preferably in at least 90% of the subjects of a specific group or population analyzed.

In another particular embodiment, the first method of the invention comprises comparing the immunoreactivity of the analyzed sample with the immunoreactivity of a second sample from the same subject in a later time period. It is therefore possible to either evaluate disease progression or to evaluate the efficacy of the treatment if said second sample has been obtained after the subject has been treated for CRC. In the sense used in this description, the term “immunoreactivity” refers to the presence or level of binding of an antibody or antibodies in a sample to one or more target antigens, for example, the ACEs of Table 2. An “immunoreactivity pattern” refers to a binding profile of antibodies in a sample (autoantibodies) to a plurality of target antigens (e.g., the ACEs of Table 2).

In another embodiment, the first method of the invention further comprises analyzing the presence of one or more additional markers of CRC, for example, CEA or autoantibodies against Pim1, SRC, MAPKAPK3, FGFR4, STK4 and/or ACVR2B proteins.

### Method for Detecting Autoantibodies

In another aspect, the invention relates to a method for detecting an autoantibody in a sample, hereinafter second method of the invention, comprising:
(a) contacting a sample with an antibody capturing entity (ACE), wherein said ACE is selected from the group consisting of:
   (i) an ACE comprising the amino acid sequence shown in SEQ ID NO: 4 or a variant thereof containing an epitope recognizable by an autoantibody;
   (ii) an ACE comprising the amino acid sequence shown in SEQ ID NO: 2 or a variant thereof containing an epitope recognizable by an autoantibody;
   (iii) an ACE comprising the amino acid sequence shown in SEQ ID NO: 6 or a variant thereof containing an epitope recognizable by an autoantibody;
   (iv) an ACE comprising the amino acid sequence shown in SEQ ID NO: 3 or a variant thereof containing an epitope recognizable by an autoantibody, wherein said ACE is not MST1 protein;
   (v) an ACE comprising the amino acid sequence shown in SEQ ID NO: 5 or a variant thereof containing an epitope recognizable by an autoantibody;
   (vi) an ACE comprising the amino acid sequence shown in SEQ ID NO: 1 or a variant thereof containing an epitope recognizable by an autoantibody; and
   (vii) any combination of said ACEs (i)-(vi); and
(b) detecting the formation of an autoantibody-ACE complex, wherein the detection of said autoantibody-ACE complex is indicative of the presence of said autoantibody in said sample.

Generally, the characteristics of said ACEs referred to in the second method of the invention are the same as the characteristics of the ACEs referred to in the first method of the invention. The techniques for detecting the autoantibodies according to the second method of the invention are the same as those mentioned in relation to the first method of the invention, so they are herein incorporated by reference.

The particular embodiments of the first method of the invention, as well as the definitions of the terms used also apply to the second method of the invention, so they are incorporated in this second method of the invention by reference.

This second method of the invention allows correlating the results obtained with those pathologies in which immune responses with the subsequent production of autoantibodies are generated. Illustrative, non-limiting examples of said pathologies include some types of cancer, e.g., CRC, hepatocellular carcinoma (Imai, H et al. Intervirology 35:73-85), breast cancer, prostate cancer (Wang X et al. N Engl J. Med. 2005; 353(12):1224-35), lung cancer, etc. and autoimmune diseases.

In a particular embodiment, the second method of the invention comprises the detection of an autoantibody against an ACE comprising the amino acid sequence shown in SEQ ID NO: 4 or a variant thereof containing an epitope recognizable by an autoantibody, and furthermore the detection of an autoantibody selected from the group consisting of:
(i) an autoantibody against an ACE comprising the amino acid sequence shown in SEQ ID NO: 2 or a variant thereof containing an epitope recognizable by an autoantibody;
(ii) an autoantibody against an ACE comprising the amino acid sequence shown in SEQ ID NO: 6 or a variant thereof containing an epitope recognizable by an autoantibody;
(iii) an autoantibody against an ACE comprising the amino acid sequence shown in SEQ ID NO: 3 or a variant thereof containing an epitope recognizable by an autoantibody;
(iv) an autoantibody against an ACE comprising the amino acid sequence shown in SEQ ID NO: 5 or a variant thereof containing an epitope recognizable by an autoantibody;
(v) an autoantibody against an ACE comprising the amino acid sequence shown in SEQ ID NO: 1 or a variant thereof containing an epitope recognizable by an autoantibody; and
(vi) any combination of autoantibodies (i) to (v).

In another particular embodiment, the second method of the invention comprises the detection of an autoantibody against an ACE comprising the amino acid sequence shown in SEQ ID NO: 1 or a variant thereof containing an epitope recognizable by an autoantibody, and furthermore the detection of an autoantibody selected from the group consisting of:
(i) an autoantibody against an ACE comprising the amino acid sequence shown in SEQ ID NO: 4 or a variant thereof containing an epitope recognizable by an autoantibody;
(ii) an autoantibody against an ACE comprising the amino acid sequence shown in SEQ ID NO: 2 or a variant thereof containing an epitope recognizable by an autoantibody;
(iii) an autoantibody against an ACE comprising the amino acid sequence shown in SEQ ID NO: 6 or a variant thereof containing an epitope recognizable by an autoantibody;
(iv) an autoantibody against an ACE comprising the amino acid sequence shown in SEQ ID NO: 3 or a variant thereof containing an epitope recognizable by an autoantibody;
(v) an autoantibody against an ACE comprising the amino acid sequence shown in SEQ ID NO: 5 or a variant thereof containing an epitope recognizable by an autoantibody; and
(vi) any combination of autoantibodies (i) to (v).

### Antibody Capturing Entity (ACE)

In another aspect, the invention relates to an antibody capturing entity (ACE), hereinafter ACE of the invention, selected from the group consisting of:
(i) an ACE comprising the amino acid sequence shown in SEQ ID NO: 4 or a variant thereof containing an epitope recognizable by an autoantibody, wherein said ACE is not SULF1 protein;
(ii) an ACE comprising the amino acid sequence shown in SEQ ID NO: 2 or a variant thereof containing an epitope recognizable by an autoantibody, wherein said ACE is not GRN protein;
(iii) an ACE comprising the amino acid sequence shown in SEQ ID NO: 6 or a variant thereof containing an epitope recognizable by an autoantibody, wherein said ACE is not GTF2i protein; and
(iv) an ACE comprising the amino acid sequence shown in SEQ ID NO: 3 or a variant thereof containing an epitope recognizable by an autoantibody, wherein said ACE is not MST1 protein;
(v) an ACE comprising the amino acid sequence shown in SEQ ID NO: 5 or a variant thereof containing an epitope recognizable by an autoantibody, wherein said ACE is not SREBF2 protein;
(vi) an ACE comprising the amino acid sequence shown in SEQ ID NO: 1 or a variant thereof containing an epitope recognizable by an autoantibody, wherein said ACE is not NHSL1 protein; and
(vii) any combination of ACEs (i) to (vi).

As previously defined, an ACE is a macromolecular entity, for example, a peptide, a protein or a phage, binding specifically to an antibody (or autoantibody). In a particular embodiment, said ACE comprises a peptide or a protein binding specifically to an antibody (or autoantibody). Said peptide can either be immobilized on a support or exposed on the phage surface. In a preferred particular embodiment, said ACE is a peptide, a protein or a phage on the surface of which said peptide or said protein is exposed. Said ACE can be immobilized on a solid support if desired.

In a particular embodiment, the ACE of the invention is selected from the group consisting of:
(i) a phage comprising the amino acid sequence shown in SEQ ID NO: 1 or a variant thereof containing an epitope recognizable by an autoantibody, wherein said amino acid sequence is exposed on the phage surface;
(ii) a phage comprising the amino acid sequence shown in SEQ ID NO: 2 or a variant thereof containing an epitope recognizable by an autoantibody, wherein said amino acid sequence is exposed on the phage surface;
(iii) a phage comprising the amino acid sequence shown in SEQ ID NO: 3 or a variant thereof containing an epitope recognizable by an autoantibody, wherein said amino acid sequence is exposed on the phage surface;
(iv) a phage comprising the amino acid sequence shown in SEQ ID NO: 4 or a variant thereof containing an epitope recognizable by an autoantibody, wherein said amino acid sequence is exposed on the phage surface;
(v) a phage comprising the amino acid sequence shown in SEQ ID NO: 5 or a variant thereof containing an epitope recognizable by an autoantibody, wherein said amino acid sequence is exposed on the phage surface;
(vi) a phage comprising the amino acid sequence shown in SEQ ID NO: 6 or a variant thereof containing an epitope recognizable by an autoantibody, wherein said amino acid sequence is exposed on the phage surface; and
(vii) any of the combinations of (i) to (vi).

Said phages can be obtained by conventional methods known by those of average skill in the art, and more specifically by means of the process described in Example 1.

The ACEs provided by this invention can be used in the detection of antibodies or autoantibodies against said ACEs in a sample, particularly against the amino acid sequences identified as SEQ ID NOs: 1-6 present in said ACEs, and the presence of said autoantibodies in said sample can be correlated with the diagnosis, prognosis, monitoring the progression, or efficacy of the treatment, of a disease, so that said autoantibodies are markers, for example, of CRC.

### Composition of the Invention

In another aspect, the invention relates to a composition, hereinafter composition 1 of the invention, comprising an ACE of the invention. As previously indicated, in a particular embodiment, said ACE can be a peptide, a protein or a phage.

In a particular embodiment, composition 1 of the invention comprises at least one ACE of the invention. In another particular embodiment, composition 1 of the invention comprises at least 2 ACEs of the invention, for example, 2, 3, 4, 5 or even the 6 ACEs of the invention.

In another particular embodiment, composition 1 of the invention comprises an ACE comprising the amino acid sequence shown in SEQ ID NO: 4 or a variant thereof containing an epitope recognizable by an autoantibody, and furthermore at least one ACE selected from the group consisting of:
(i) an ACE comprising the amino acid sequence shown in SEQ ID NO: 2 or a variant thereof containing an epitope recognizable by an autoantibody;
(ii) an ACE comprising the amino acid sequence shown in SEQ ID NO: 6 or a variant thereof containing an epitope recognizable by an autoantibody;
(iii) an ACE comprising the amino acid sequence shown in SEQ ID NO: 3 or a variant thereof containing an epitope recognizable by an autoantibody;
(iv) an ACE comprising the amino acid sequence shown in SEQ ID NO: 5 or a variant thereof containing an epitope recognizable by an autoantibody;
(v) an ACE comprising the amino acid sequence shown in SEQ ID NO: 1 or a variant thereof containing an epitope recognizable by an autoantibody; and
(vi) any combination of ACEs (i) to (v).

In another particular embodiment, composition 1 of the invention comprises an ACE comprising the amino acid sequence shown in SEQ ID NO: 1 or a variant thereof containing an epitope recognizable by an autoantibody, and furthermore at least one ACE selected from the group consisting of:
(i) an ACE comprising the amino acid sequence shown in SEQ ID NO: 2 or a variant thereof containing an epitope recognizable by an autoantibody;
(ii) an ACE comprising the amino acid sequence shown in SEQ ID NO: 3 or a variant thereof containing an epitope recognizable by an autoantibody;
(iii) an ACE comprising the amino acid sequence shown in SEQ ID NO: 4 or a variant thereof containing an epitope recognizable by an autoantibody;
(iv) an ACE comprising the amino acid sequence shown in SEQ ID NO: 5 or a variant thereof containing an epitope recognizable by an autoantibody;
(v) an ACE comprising the amino acid sequence shown in SEQ ID NO: 6 a variant thereof containing an epitope recognizable by an autoantibody; and
(vi) any combination of ACEs (i) to (v).

In another particular embodiment, composition 1 of the invention comprises at least one ACE of the invention and at least one protein selected from the group consisting of SULF1 protein or a variant thereof, MST1 protein or a variant thereof, and their combinations. The person skilled in the art will note that it is possible to use SULF1 or MST1 proteins of different species; nevertheless, in a preferred embodiment, composition 1 of the invention includes SULF1 or MST1 proteins of a human origin, such as human SULF1 protein, with NCBI database accession number (May 1, 2011 version) EAW86954.1 and its amino acid sequence is SEQ ID NO: 10, and human MST1 protein with NCBI database accession number (May 1, 2011 version) AAA83254.1 and its amino acid sequence is SEQ ID NO: 9. The term “variant” has already been defined above in the “Definitions” section.

In another particular embodiment, composition 1 of the invention comprises:
a) an ACE selected from the group consisting of:
   (i) an ACE comprising the amino acid sequence shown in SEQ ID NO: 4 or a variant thereof containing an epitope recognizable by an autoantibody,
   (ii) an ACE comprising the amino acid sequence shown in SEQ ID NO: 2 or a variant thereof containing an epitope recognizable by an autoantibody;
   (iii) an ACE comprising the amino acid sequence shown in SEQ ID NO: 6 or a variant thereof containing an epitope recognizable by an autoantibody;
   (iv) an ACE comprising the amino acid sequence shown in SEQ ID NO: 3 or a variant thereof containing an epitope recognizable by an autoantibody;
   (v) an ACE comprising the amino acid sequence shown in SEQ ID NO: 5 or a variant thereof containing an epitope recognizable by an autoantibody;
   (vi) an ACE comprising the amino acid sequence shown in SEQ ID NO: 1 or a variant thereof containing an epitope recognizable by an autoantibody; and
   (vii) any combination of ACEs (i) to (vi); and
b) a protein selected from the group consisting of SULF1 protein or a variant thereof, MST1 protein or a variant thereof, and their combinations.

In a preferred embodiment, said composition 1 of the invention comprises:
a) an ACE selected from the group consisting of:
   (i) an ACE comprising the amino acid sequence shown in SEQ ID NO: 2 or a variant thereof containing an epitope recognizable by an autoantibody,
   (ii) an ACE comprising the amino acid sequence shown in SEQ ID NO: 6 or a variant thereof containing an epitope recognizable by an autoantibody;
   (iii) an ACE comprising the amino acid sequence shown in SEQ ID NO: 5 or a variant thereof containing an epitope recognizable by an autoantibody;
   (iv) an ACE comprising the amino acid sequence shown in SEQ ID NO: 1 or a variant thereof containing an epitope recognizable by an autoantibody; and
   (v) any combination of ACEs (i) to (iv); and
b) a protein selected from the group consisting of SULF1 protein or a variant thereof, MST1 protein or a variant thereof, and their combinations.

This composition 1 of the invention has provided good results in the diagnosis of CRC [Example 4].

In another aspect, the invention relates to a composition, hereinafter composition 2 of the invention, comprising SULF1 protein or a variant thereof and MST1 protein or a variant thereof. The characteristics of said SULF1 and MST1 proteins, and of their variants, have been mentioned previously.

In a particular embodiment, composition 2 of the invention further comprises at least one ACE of the invention. In another particular embodiment, composition 2 of the invention comprises at least 2 ACEs of the invention.

In another particular embodiment, said composition 2 of the invention comprises:
a) SULF1 protein or a variant thereof;
b) MST1 protein or a variant thereof; and
c) an ACE selected from the group consisting of:
   (i) an ACE comprising the amino acid sequence shown in SEQ ID NO: 2 or a variant thereof containing an epitope recognizable by an autoantibody,
   (ii) an ACE comprising the amino acid sequence shown in SEQ ID NO: 6 or a variant thereof containing an epitope recognizable by an autoantibody;
   (iii) an ACE comprising the amino acid sequence shown in SEQ ID NO: 5 or a variant thereof containing an epitope recognizable by an autoantibody;
   (iv) an ACE comprising the amino acid sequence shown in SEQ ID NO: 1 or a variant thereof containing an epitope recognizable by an autoantibody; and
   (v) any combination of ACEs (i) to (iv).

In a particular embodiment, both composition 1 of the invention and composition 2 of the invention are supported on a solid support.

### Kit of the Invention and Applications

In another aspect, the invention relates to a kit, hereinafter kit 1 of the invention, comprising a composition of the invention. In a particular embodiment, said composition of the invention is composition 1 of the invention. In another particular embodiment, said composition of the invention is composition 2 of the invention.

In another aspect, the invention relates to the use of kit 1 of the invention for:
detecting an antibody in a sample,
detecting an autoantibody in a subject suspected of having colorectal cancer (CRC),
diagnosing whether a subject has CRC,
determining the risk of a subject developing CRC,
monitoring CRC progression in a subject,
evaluating the efficacy of a treatment against CRC, or
predicting survival of a subject who has CRC.

For said applications, kit 1 of the invention will include the reagents necessary for detecting autoantibodies against at least one ACE of Table 2.

In a particular embodiment, kit 1 of the invention comprises an ACE of the invention. In another particular embodiment, the kit of the invention comprises composition 1 of the invention. In another particular embodiment, the kit of the invention comprises composition 2 of the invention. Therefore, in a particular embodiment, kit 1 of the invention comprises a protein selected from the group consisting of SULF1 protein or a variant thereof, MST1 protein or a variant thereof, and their combinations.

In another aspect, the invention relates to a kit, hereinafter kit 2 of the invention, comprising a reagent for detecting SULF1 protein or a variant thereof. In a particular embodiment, said kit 2 of the invention further comprises a reagent for detecting MST1 protein or a variant thereof. In another particular embodiment, kit 2 of the invention comprises an ACE selected from the group of ACEs mentioned in Table 2.

Kit 2 of the invention can be used in the same applications as kit 1 of the invention.

Both kit 1 of the invention and kit 2 of the invention can further contain all those reagents necessary for detecting the amount of autoantibodies against the ACEs defined previously, or against the SULF1, MST1 proteins or their variants, such as but not being limited to the following for example
secondary antibodies labeled with a marker specifically recognizing the autoantibody-ACE complexes;
substrates for the markers present in said labeled secondary antibodies; and
positive and/or negative controls.

Likewise, said kits 1 and 2 of the invention can further include, without any type of limitation, buffers, agents for preventing contamination, protein degradation inhibitors, etc. In addition, the kit of the invention can include all the supports and containers necessary for being put into practice and for optimization. Preferably, the kit further comprises instructions for use.

### Method for the Diagnosis of CRC

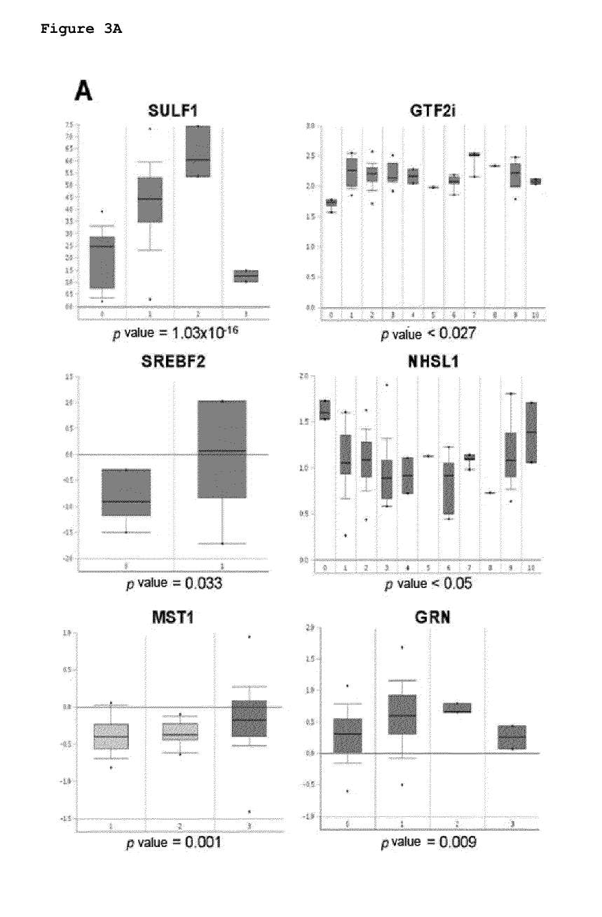

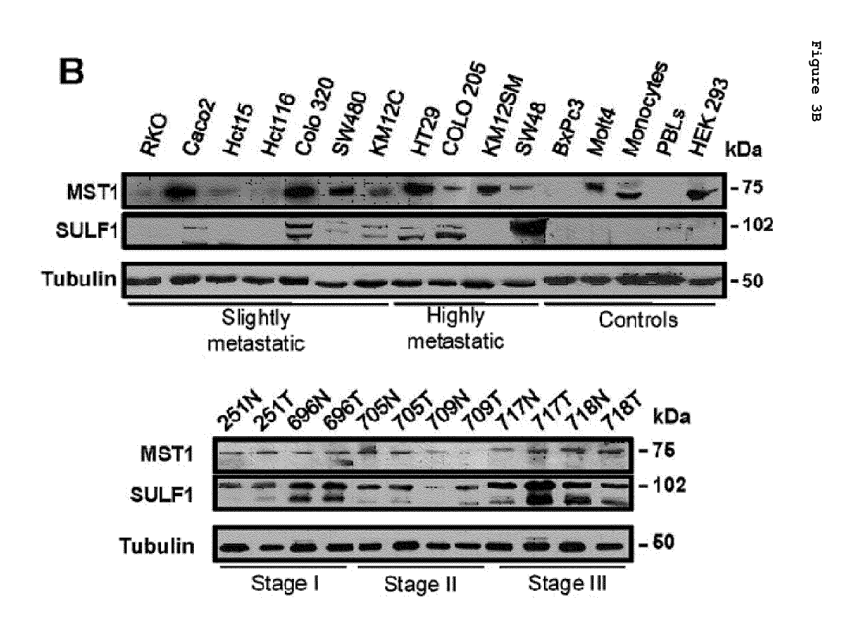

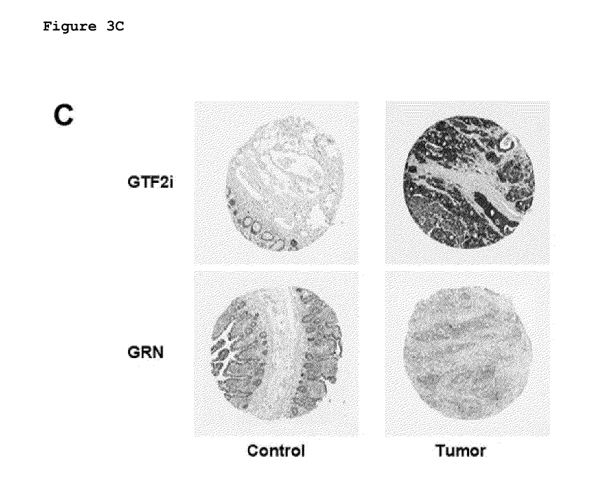

The authors of the present invention have additionally found that the overexpression of SULF1 protein is correlated with CRC, as shown in FIG. 3.

Therefore, in another aspect, the invention relates to a method for diagnosing whether a subject suffers colorectal cancer (CRC), hereinafter third method of the invention, comprising determining SULF1 protein level in a sample from said subject, wherein if said SULF1 protein level is greater than the SULF1 protein level of a reference sample, it is indicative of the subject having CRC.

For putting the third method of the invention into practice, the sample can preferably be a tissue sample, such as a colon or tumor tissue sample.

The term “diagnosis” has already been defined above.

The methods for determining protein level (concentration) are well-known by a person skilled in the art and include a number of alternatives. Virtually any method which allows determining (quantifying) SULF1 protein level can be used in putting the third method of the invention into practice.

In a particular embodiment, SULF1 protein level is quantified by means of a conventional method allowing detecting and quantifying said protein in a sample to be studied, such as a sample from a subject. By way of non-limiting illustration, said SULF1 protein level can be determined by means of an immunoassay, for example, ELISA, etc., by means of nuclear magnetic resonance (NMR) or by means of any other suitable technique known in the state of the art. In a preferred embodiment, protein level is determined by means of an immunoassay. In a preferred particular embodiment, said immunoassay is an immunoblot (Western blot or membrane immunodetection). To that end, briefly, a protein extract is obtained from a biological sample isolated from a subject and the protein is separated by electrophoresis in a support medium capable of retaining it. Once the proteins are separated, they are transferred to a different support or membrane where they can be detected by using specific antibodies recognizing the protein in question (SULF1). Said membrane is hybridized with a first specific antibody (or primary antibody) recognizing SULF1 protein. Then the membrane is hybridized with a second antibody (or secondary antibody) specifically recognizing the primary antibody and conjugated or bound with a marker compound. In an alternative embodiment, the antibody recognizing a SULF1 protein is conjugated or bound to a marker compound, and the use of a secondary antibody is not necessary. Different formats, supports and techniques that can be used for performing this preferred aspect of the third method of the invention are known.

In another preferred particular embodiment, the immunoassay comprises an immunohistochemical assay. Immunohistochemistry techniques allow the identification of characteristic antigenic determinants in tissue and cytology samples. Analysis by means of immunohistochemistry (IHC) is performed on tissue sections, either frozen or included in paraffin, from a biological sample isolated from a subject. These sections are hybridized with a specific antibody or primary antibody recognizing specific antibodies recognizing a SULF1 protein. The sections are then hybridized with a secondary antibody capable of specifically recognizing the primary antibody and is conjugated or bound to a marker compound. In an alternative embodiment, the antibody recognizing SULF1 protein is conjugated or bound to a marker compound, and the use of a secondary antibody is not necessary.

By way of non-limiting illustration, “SULF1 protein level” refers but is not limited to a quantifiable, semiquantifiable, or relative amount of said SULF1 protein, as well as to any other value or parameter related to said protein or which can be derived therefrom. Said values or parameters comprise signal intensity values obtained from any of the physical or chemical properties of said protein obtained either by means of direct measurement, e.g., intensity values of mass spectroscopy, nuclear magnetic resonance, etc., or by means of indirect measurement, e.g., by means of any of the systems of measurement described herein, for example, by means of the measurement obtained from a secondary component or a biological measurement system (e.g., the measurement of cell responses, ligands, “tags” or enzymatic reaction products). The SULF1 protein level determined in a sample, such as a biological sample from the subject subjected to study, is said to be “greater” than the reference level of said SULF1 protein when, according to the invention, the level of said protein in the biological sample to be analyzed is at least 1.5 times, 5 times, 10 times, 20 times, 30 times, 40 times, 50 times, 60 times, 70 times, 80 times, 90 times, 100 times or even more, with respect to the reference level of said protein.

As it is used herein, the term “reference level” generally refers to the level of a product, for example, SULF1 protein, present in control subjects. In a particular embodiment, said control subjects are subjects who do not suffer a specific disease (e.g., CRC), whereas in another particular embodiment, said control subject is the actual subject under study, which is particularly useful for evaluating the tracking of a disease (e.g., CRC) or for evaluating the efficacy of a treatment for said disease (e.g., CRC), etc., for which the reference level of a given product can be the level of said product determined in a sample from the same subject under study but taken days, weeks, months or even years before for the purpose of evaluating the tracking of the disease, or taken before, for example, the application in the subject of a treatment for said disease for the purpose of evaluating its efficacy.

Due to the variability that can occur between the different subjects in terms of the production of SULF1 protein, the reference level could be obtained from a set of samples from a population of healthy subjects (e.g., subjects who do not suffer CRC) and by calculating the mean level of the product in question (SULF1 protein) in said population of healthy subjects.

The reference level of a certain product, for example, SULF1 protein, can be determined from a reference sample which can be analyzed, for example, simultaneously or consecutively, together with the sample to be analyzed (test sample). The reference level can generally be derived from the normal distribution limits of a physiological amount found in a population of control subjects. Said physiological amount can be determined by several well-known techniques, as described in this description.

According to the present invention, said reference level allows discriminating the presence of CRC and can therefore be used in the diagnosis, prognosis or tracking of CRC progression.

The markers and methods provided by the present invention are suitable for diagnosing CRC, as well as for predicting the development of a CRC, monitoring the progression of a CRC and/or evaluating the efficacy of treatment administered to a subject who has CRC.

Assays performed by the inventors have clearly shown that the phages identified as MST1, SULF1, NHSL1, SREBF2, GRN and GTF2i allow discriminating between tumor sera (CRC) and control sera by ELISA. Although sensitivity was relatively low for the individual phages, ranging between 46% and 60%, specificity was greater, between 50% and 73.9%, and furthermore the combination of the 6 phages as a predictor of CRC provided an AUC of 0.82 with sensitivity and specificity of 70% and 73.9% [Example 4].

In turn, the combination of SULF1 and MST1 recombinant proteins together with NHSL1, GRN, SREBF2 and GTF2i phages allowed predicting the disease (CRC) with 72% sensitivity and 87% specificity, with an AUC of 0.83. If the age of the patient is further taken into account, the AUC is of 0.91. These markers further allow grouping results by discriminating not only sick individuals, but also different stages of the disease. Likewise, autoantibodies against MST1 and NHSL1 are associated with the clinical prognosis of CRC patients. Therefore, the detection of this panel of autoantibodies in serum is therefore a simple and non-invasive method for the diagnosis/prognosis of CRC.

It has additionally been observed that SULF1 protein overexpression is correlated with the diagnosis of CRC [FIG. 3].

### EXAMPLES

The invention will be illustrated below by means of assays conducted by the inventors, which clearly show the specificity and effectiveness of the method for the diagnosis/prognosis of CRC based on the detection in serum of antibodies against the tumor autoantigens described.

### Example 1

### Analysis of Sera from CRC Patients with Microarrays Printed with T7 Phages

### I. Materials and Methods

### Crc and Reference Sera

The sera used in microarray and survival analyses were obtained from patients at Hospital Universitario de Bellvitge, Instituto Catalan de Oncologia de Barcelona, Hospital Puerta de Hierro de Madrid and Hospital de Cabue n̅es de Gijón, after obtaining the written consent of all the patients included in the study.

3 sera from CRC patients with Duke's stage B, 3 with stage C and 6 with stage D (3 with liver metastasis and 3 with lung metastasis) were used to select the libraries of CRC-specific T7 phages. 15 sera from CRC patients with different stages, with a mean age of 66.3 years (age range 54-82) and 15 sera from control individuals with a similar mean age and the same sex ratio as the CRC patients were selected for microarray screening.

For the survival analysis, another panel of 95 CRC sera with over 10 years of tracking was tested. The median age was 66.2 years (range between 23-90 years). The clinical data of all the patients are included in Table 3. All the samples were managed anonymously according to the ethical and legal standards of the Consejo Superior de Investigaciones Cientificas (CSIC) (Superior Council for Scientific Research).

TABLE 3
Clinical-pathological information of patients whose serum was used
for the identification and validation of the autoantibodies.

| CRC patients (n) | 160 | Healthy donors (n) | 61 |
|---|---|---|---|
| Mean age (years) | 67.7 | Mean age (years) | 61.7 |
| Age range (years) | 23-91 | Age range (years) | 34-89 |
| Sex: | | Sex: | |
| Male | 65.6% | Male | 60.7% |
| Female | 34.4% | Female | 39.3% |
| Duke's stage: | | | |
| I | 40.6% | | |
| II | 19.4% | | |
| III | 15.6% | | |
| IV | 24.4% | | |
| Prognosis: | | | |
| Dead | 33.1% | | |
| Alive | 60% | | |
| Unknown | 6.9% | | |
| Mean survival time (months) | 57.5 | | |

An independent group of sera was used for validation; 50 CRC sera representative of all Duke's stages (A-D), 46 control sera, 10 asymptomatic patients with family history, 2 with hyperplastic polyps, 2 with ulcerative colitis and 43 sera of other types of cancer (bladder, breast, lung, pancreas and stomach).

The sera used were processed in the same manner in the different hospitals; the blood samples were left at room temperature for at least 30 minutes (and a maximum of 60 minutes) to allow clot formation. The samples were subsequently centrifuged at 3000 g at 4° C. for 10 minutes. The sera were frozen and stored at −80° C. until use.

Synthesis of T7 Phage cDNA Library and Selection Rounds

The total RNA of 3 tumor tissues from CRC patients in Duke's stages A (samples A) and 3 with stage C (samples C), was isolated by means of the reagent Trizol (Invitrogen). 4 μg of each RNA were used, mixing samples A and samples C separately, for the synthesis of cDNA. The T7 phage cDNA libraries of CRC in were constructed using the OrientExpress cDNA synthesis and cloning system (Novagen) according to the manufacturer's instructions. The cDNA was synthesized by means of RT-PCR using the oligonucleotide oligo(dT). The cloning was performed indistinctly in vectors T7Select 415-1 and T7Select 10-3b, which differ in the size of the insert they allow. Phage growth was obtained in E. coli strains BL21 and BLT 5403, respectively. The four T7 libraries were titrated making serial dilutions of the T7 phages in Petri dishes. The sizes of the libraries were greater than 10⁶ pfu/mL in all cases. The phage selection rounds were performed using on one hand the combination of the 2 libraries constructed with the T7Select 415-1 vector and on the other the combination of the 2 libraries constructed with the T7Select 10-3b vector.

Negative selection was performed first. To that end, protein A/G coupled magnetic particles (Invitrogen) were incubated with a mixture of 8 control sera (120 μL of mixture of control sera, diluted 1:50, at 4° C. overnight) to bind the IgGs of the control subjects. The phages were subsequently incubated with said magnetic particles to remove those phages bound to the IgGs of the control sera. Secondly, 4 mixtures of sera (stage B mixture: from 3 CRC patients with Duke's stage B, stage C mixture: from 3 patients with Duke's stage C, stage D-H mixture: from 3 patients with Duke's stage D and liver metastasis and stage D-P mixture: from 3 patients with Duke's stage D and lung metastasis) were incubated with protein A/G coupled magnetic particles to enrich the phage libraries with CRC specific phages. The phages not retained in the negative selection were incubated with the magnetic particles previously incubated with the different mixtures of sera from CRC patients. The phages bound to said magnetic particles were eluted with 100 μL of 1% SDS and amplified in E. coli BLT5406 or BL21. A total of 4 selection rounds were conducted to enrich T7 phage libraries with CRC specific phages. Individual clones of the 8 selections which were printed in nitrocellulose microarrays were finally amplified.

### Phage Microarrays

After the amplification of monoclonal phages, the bacteria lysates were centrifuged and the supernatants containing the phages were diluted 1:2 in PBS with 0.1% Tween 20 (PBST) for printing in duplicate in nitrocellulose microarrays (Whatman/Schleicher & Schuell's) with the OmniGrid Spotter robot (GeneMachines, San Carlos, Calif.). The negative controls used in the printing were BSA (Sigma Aldrich), printing buffer or empty gaps. T7 and human IgG protein (Sigma-Aldrich) were printed as positive controls to verify the quality of the array.

Thirty sera (15 from CRC patients and 15 from normal individuals) were incubated with the phage microarrays as described previously (Chaterjee, M et al. 2006. Cancer Res. 66:1181-1190). Briefly, the slides were equilibrated with PBS at room temperature for 5 minutes and blocked with 3% skim milk in PBS (3% MPBS) for 1 hour at room temperature under stirring. The arrays were subsequently incubated with 6.6 μL of human serum (dilution 1:300), 120 μg of E. coli lysate and 0.3 μg of anti T7-tag monoclonal antibody (Novagen) diluted in 2 mL of 3% MPBS for 90 minutes at room temperature. The slides were washed 3 times with PBST for 10 minutes to eliminate non-specific binding and were incubated with an AlexaFluor 647-conjugated anti-human IgG antibody (Invitrogen) diluted 1:2,000 and an AlexaFluor 555-conjugated anti-mouse IgG antibody (Invitrogen) diluted 1:40,000 in 3% MPBS for detecting the human antibodies bound to T7 phages and the T7 phages, respectively. Subsequently, the microarrays were washed 3 times with PBST, once with PBS and were dried by means of centrifugation at 1200 rpm for 3 minutes. Finally, the slides were scanned in a ScanArray™5000 (Packard BioChip Technologies). The Genepix Pro 7 image analysis program (Axon Laboratories) was used to quantify the intensity of the points.

### Statistical Analysis

The microarray data were normalized and processed using the Asterias applications (http://asterias.bioinfo.cnio.es/), an interface for using software packages, Limma and marrayNorm from Bioconductor. After applying a background noise correction and global Loess normalization (http://dnmad.bioinfo.cnio.es/), the data were processed to filter the missing values or points with too high of a variance, to combine duplicates and then obtain a single log transformed base 2 value for each phage (http://prep.bioinfo.cnio.es/). The groups of CRC patients and healthy individuals were compared by performing a t-test with the Pomelo II program (http://pomelo2.bioinfo.cnio.es/), where p values were obtained by means of 200000 permutations. The Pomelo II program generated a graph showing the phages with a positive result false discovery rate (FDR) value below 0.15 and an unadjusted p value below 0.05.

The bootstrapping analyses were adjusted by means of a logistic regression model where the probability of being a tumor against the probability of being normal was modeled as a function of the variables (phages and proteins). The age and sex of the patients were also included in the model to correct the possible effects of these variables. The area under the ROC curve (AUC) was calculated to evaluate the predictive ability of the models. The AUC calculated directly with the original model and the complete data set is biased towards high values. Therefore, bootstrapping with 1,000 replicate samples was used to obtain a corrected AUC not biased towards high values, giving an estimate of the AUC that could be obtained with an independent future validation (Efron B. J. Am. Stat Assoc. 1983; 78:316-331). The models were adjusted using the Harrell design library (Harrel F. Springer. 2001) with the statistical computing system R (Team RDC, 2009).

### II. Results

The tissue RNA of CRC from 6 patients (three with Duke's stage A and three with Duke's stage C) was used to construct T7 phage libraries containing cDNA fragments in 2 vectors (T7Select 415-1 or T7select 10-3b). After selecting CRC specific phages 8 different libraries enriched in tumor specific phages, depending on the vector and the mixture of sera (B, C, H and P) used during selection, were obtained. A total of 1,536 individual phages were amplified (192 individual phages of each selection) and were printed in duplicate in nitrocellulose arrays. An anti-T7 antibody which allowed observing the presence of a homogenous signal in the array was used as a control of the amount of phage printed in the array. The intensity of the 2 points corresponding to the same phage within the same array and between two different arrays was represented for the purpose of determining intra- and inter-array reproducibility. It was determined that intra- and inter-array reproducibility was good with R2 values of 0.9703 and 0.9091, respectively.

The arrays with 30 sera (15 from patients with different stages of CRC and 15 from healthy controls) were incubated to evaluate the immune response in CRC patients. After quantifying the images and normalizing the data, the signal of the tumor sera was compared with the healthy sera using a t-test with 200,000 permutations. Between the 2 groups 128 phages showed different reactivities, with an FDR <0.22. Out of those phages, 78 showed increased reactivity in CRC whereas 50 had reduced reactivity in sera with CRC. The representation of the supervised analysis of the 45 phages with the lowest FDR (<0.15) showed a clear separation between CRC patients and healthy individuals.

### Example 2

### Identification of the Phage-Displayed Sequences

### I. Materials and Methods

### Sequencing and Analysis of the Internal Sequences by Means of BLASTp

The DNA inserted in the phage genome was amplified by PCR using forward primer T7_up2: 5′-TGCTAAGGACAACGTTATCGG-3′ (SEQ ID NO:13) and reverse primer T7_down2: 5′-TTGATACCGGACGTTCAC-3″ (SEQ ID NO:14). The PCR products were precipitated with ethanol and sequenced directly with forward primer T7_up2.

A search was conducted in the NCBI database with BLASTp software to find sequence homology for each peptide displayed on the selected phage surface.

### Proteins, Antibodies and Cell Lines

MST1/STK4 and SULF1 human recombinant proteins were expressed in E. coli. MST1 cDNA was sub-cloned into pET28a vector (Novagen). SULF1 cDNA was cloned into pDONR221 vector and subsequently into pDEST17 expression vector. The 2 6xHis-MST1 and 6×His-SULF1 fusion proteins were expressed in E. coli strain BL21 (DE3) and purified to homogeneity by means of HisTrap column affinity chromatography (GE Healthcare). Finally, the proteins were dialyzed against PBS and concentrated. EBNA1 protein used as a positive control in ELISA assays was purchased from the company Tebu-Bio.

Antibodies against MST1/STK4, SULF1 and tubulin used in the membrane immunodetection were purchased from the companies Atlas antibodies, Santa Cruz Biotechnology and Sigma, respectively. The TrueBlot peroxidase-conjugated anti-rabbit IgG antibody was purchased from the company eBioscence and the peroxidase-conjugated anti-mouse IgG and anti-human IgG antibodies were purchased from DakoCytomation.

Colorectal cancer cell lines RKO, Caco2, Hct15, Hct116, Colo320, SW480, SW48, KM12C, KM12SM, HT29, Colo205 and reference cell lines (HEK293 and MOLT4) were grown according to protocols established. Peripheral blood lymphocytes (PBL) and monocytes were isolated from a healthy donor.

### Western-Blot Analysis

The preparation of the cell lines and paired tissue extracts was performed according to the following protocol. The cells and the tissues were washed twice with PBS before lysis with 500 μL of 0.5% SDS with protease inhibitors (Roche Applied Science). The concentration of the extracts was determined by means of the 2D-Quant kit (GE Healthcare) after clarifying the sample by means of centrifugation at 12,000 g for 15 minutes at 4° C.

25 μg of protein extract were separated in 10% SDS-PAGE gel and transferred to nitrocellulose membranes (Hybond-C Extra) according to established protocols (Babel et al. Mol. Cell. Proteomics 2009; 8:2382-95). The membrane was blocked with 3% MPBS and incubated overnight at 4° C. with the antibodies against MST1 (1:1,000 dilution), SULF1 (1:3,000 dilution) or tubulin (1:5,000 dilution). Immunodetection was performed using an HRP-conjugated anti-mouse IgG antibody (1:5,000 dilution) or an HRP-conjugated anti-rabbit IgG antibody (1:5,000 dilution). Antibody binding was finally detected using ECL (GE Healthcare) or SuperSignal Femto (Pierce).

### ELISA

T7 phage capture ELISA plates (Novagen) were blocked for 2 hours at 37° C. with 3% MPBS and incubated overnight with 100 μL of the bacterial lysate of the phages diluted in 3% MPBS. After washing 3 times with PBST, the plates were blocked with 3% MPBS for 1 hour at 37° C. and incubated with 100 μL of human serum (1:50 dilution in 3% MPBS) for 1 hour at 37° C. After 3 additional washes, peroxidase-labeled anti-human IgG antibody (1:3,000 in 3% MPBS) was added for 2 hours at room temperature. The signal was detected with 3,3′,5,5′-tetramethylbenzidine substrate (Sigma) for 10 minutes, stopping the reaction with 1 M HCl and measuring the signal at 450 nm.

The competitive assay between the peptides displayed on phage surfaces and the recombinant proteins was performed using the T7 phage capture plates (Novagen) following the preceding protocol, except the human sera were pre-incubated overnight at 4° C. with serial dilutions of MST1, SULF1 or GST proteins. The sera thus pre-incubated were tested against EBNA1 in ELISA plates (Maxisorp, Nunc) as a positive control to verify that the competition for IgGs between the phage and its respective recombinant protein was specific.

The ELISAs with MST1, SULF1 and EBNA1 proteins were performed as described previously (Babel et al. Mol. Cell. Proteomics 2009; 8:2382-95). The concentration of CEA in the sera from CRC patients and the control sera was determined by means of a specific immunological test following manufacturer's recommendations (MP Biomedicals).

### Statistical Analysis

The ELISA data for each individual marker (full length phage or protein) were evaluated calculating an ROC curve (receiver operating characteristic curve). The corresponding area under the curve (AUC) was calculated using the JMP7 program (SAS). The mean and standard deviation of the immunohistochemistry results were calculated using the Microsoft Office Excel 2007 program. The one-tailed Student's t-test was carried out using the immunohistochemistry results, assuming that the unequal variances for determining the means of the normal and tumor groups were significantly different from one another.

### II. Results

Forty-three unique amino acid sequences were obtained fused to T7 phage capsid protein 10B among the 78 phages showing increased reactivity in sera from CRC patients.

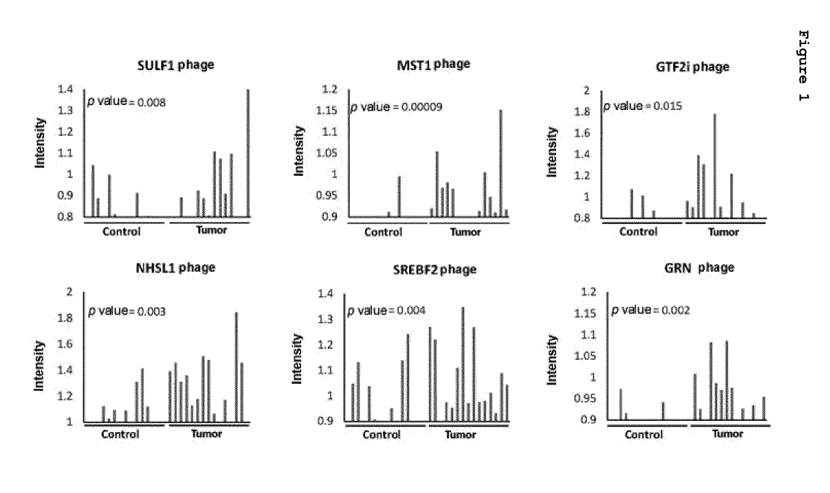

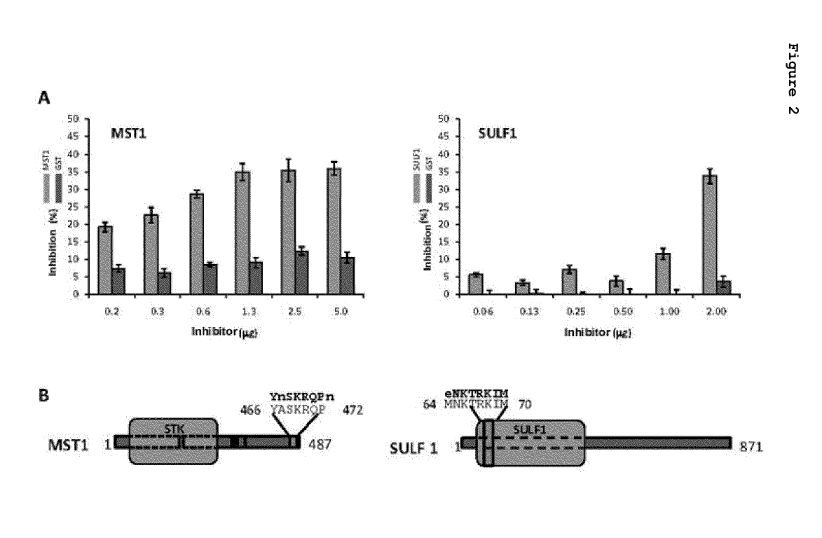

Out of the 43 unique phages those phages which contained between 8 and 20 residues with high homology to known protein sequences, which appeared a greater number of times with the same amino acid sequence and which had a low p value, were selected to verify the results. Sequences homologous to MST1/STK4, SULF1, NHSL1, SREBF2, GRN and GTF2i proteins were identified. All of them had a significantly greater signal in the microarray with the serum from CRC patients than with the control sera (FIG. 1). The phages were identified by the name of the protein with which homologous sequences were identified. MST1/STK4 protein was identified previously as a tumor-associated antigen in CRC using commercial protein microarrays (Babel et al. Mol. Cell. Proteomics 2009; 8:2382-95) and the SULF1 gene was described as being overexpressed in a transcriptome analysis of CRC (Madoz-Gurpide et al. Mol Cell Proteomics, 2006; 5:1471-83).

A competitive IgG assay was performed between the phages and SULF1 and MST1 human recombinant proteins for the purpose of confirming that the phage-displayed peptides the homologous sequence of which belonged to SULF1 and MST1 proteins. The binding of the immunoglobulins present in the human sera to the 2 phages was inhibited in a dose-dependent manner with MST1 and SULF1 recombinant proteins (FIG. 2A). GST did not affect the binding of IgGs to phages (negative control). As a specific inhibition control, it was observed that the binding of the antibodies of the patients to EBNA protein was not affected by incubation with MST1 or SULF1 proteins.

In addition, it was determined that the sequences of the phage-displayed peptides were located in the C-terminal region of MST1 and in the N-terminal region of SULF1 (FIG. 2B).

All these results confirm that the displayed peptides correspond to immunodominant epitopes of MST1 and SULF1 proteins.

### Example 3

### The Identified Proteins are Overexpressed in Colorectal Cancer

Tumor-associated antigens recognized by autoantibodies are generally overexpressed in cell lines and in tumor tissues. Meta analysis of mRNA expression levels of the homologous proteins corresponding to the 6 phages selected [MST1/STK4, SULF1, NHSL1, SREBF2, GRN and GTF2i] was performed with the Oncomine microarray database (Rhodes et al. Neoplasia 2004; 6:1-6) (FIG. 3A). It was found that SULF1 was the most overexpressed gene in colon cancer, followed by GTF2i, MST1, GRN, NHSL1 and SREBF2. In addition, membrane immunodetection was performed with the antibodies against MST1 and SULF1 using CRC cell lines and tumor tissues from CRC patients representing the different stages of disease progression (FIG. 3B). It was found that MST1 and SULF1 proteins were expressed in most colon cancer cell lines. The greater expression of SULF1 was observed in metastatic cell lines (SW48, HT29 and COLO205) and in CRC tumor tissue in late stages.

The cellular expression patterns of the selected proteins were characterized by means of immunohistochemistry (TMA) using independent CRC tumors arranged in microarrays or by means of meta analysis of tissue microarray data obtained from the Human Protein Atlas (Berglund et al. Mol Cell Proteomics. 2008; 7:2019-27) (FIG. 3C). In all cases, more abundant expression of the protein studied was detected in tumor tissues.

Therefore, there is good correlation between the presence of autoantibodies, the abundance of proteins and gene expression.

### Example 4

### Validation of the Predictor Formed by Phage-Displayed Peptides and Their Homologous Proteins

An independent set of 96 serum samples (50 with colorectal cancer with 19 samples in early stages (A+B) and 46 healthy controls) were used for the validation of the results. MST1, SULF1, NHSL1, SREBF2, GRN and GTF2i phages were tested for their ability to discriminate between tumor sera and control sera by ELISA. ROC curves were constructed for each of the markers with the ELISA results. While sensitivity was relatively low for individual phages, ranging between 46% and 60%, specificity was higher, between 50% and 73.9%. The data was fitted to a logistic curve performing linear regressions and producing different models with different combinations of phages to investigate if different combinations of phages showed greater precision in discriminating healthy individuals from cancer patients. Therefore, the result of the combination of the 6 phages as a predictor of CRC gives an AUC of 0.82 with sensitivity and specificity of 70% and 73.9%, respectively (Table 4).

TABLE 4
Data from the ROC curves obtained from the ELISA values of the
validation of both individual phages and of combined phages.

| Phage- | | | |
|---|---|---|---|
| displayed | Specificity | Sensitivity | |
| peptide | (%) | (%) | AUC |
| SULF1 | 73.9 | 50.0 | 0.63 |
| NHSL1 | 50.0 | 56.0 | 0.59 |
| MST1 | 71.7 | 46.0 | 0.58 |
| GTF2i | 52.2 | 60.0 | 0.57 |
| SREBF2 | 69.6 | 54.0 | 0.61 |
| GRN | 50.0 | 58.0 | 0.53 |
| Combination of | 73.9 | 70.0 | 0.82 |
| 6 phages | | | |

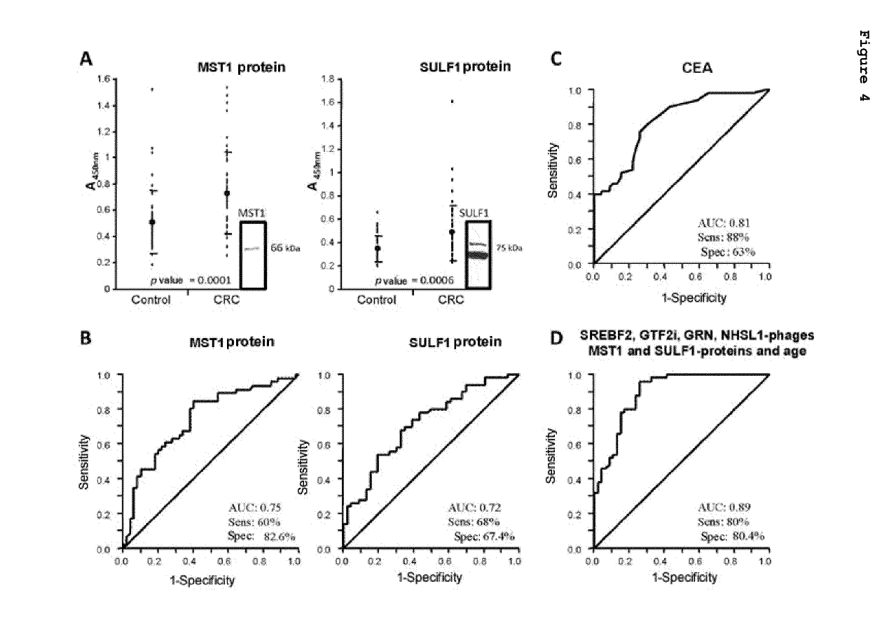

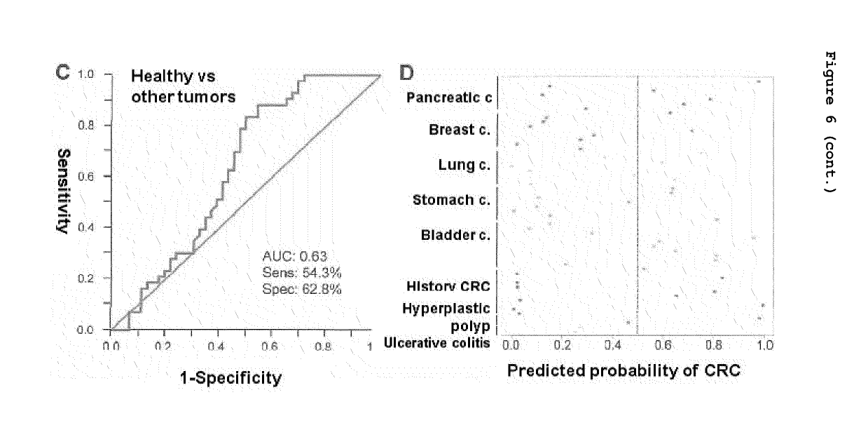

The following step consisted of seeing if the replacement of the phages with their MST1 and SULF1 recombinant proteins would improve the discriminatory power of the model (FIG. 4). The results confirmed a significant improvement of the prediction using the recombinant proteins, with AUCs of 0.71 and 0.74 for SULF1 and MST1 proteins against 0.63 and 0.58 of the respective phages (Table 2). By combining the two proteins (SULF1 and MST1) and the four phages (NHSL1, SREBF2, GRN and GTF2i), the AUC increased to 0.86 with sensitivity of 82.6% and specificity of 70% (FIG. 6A). The CEA values were lower (AUC 0.81) and when combined with the remaining predictions they barely improved the model (AUC 0.89). Different AUC estimations were further performed in the validation step to compare not only CRC versus healthy, but also CRC versus reference sera and healthy versus other tumors (FIG. 6). The most relevant result was the capacity of the model to discriminate not only CRC from healthy sera (AUC 0.86) (FIG. 6A), but also CRC from all the reference sera, which included other colon-related pathologies (AUC 0.85) (FIG. 6B). Notably, the panel seemed to not suitably discriminate healthy controls from other tumors (AUC 0.63) (FIG. 6C). The panel further seemed to significantly discriminate healthy controls from asymptomatic patients with a family history of CRC (AUC 0.78).

### Bootstrapping Analysis

Bootstrapping was also performed to obtain the corrected AUC. The initial model included linear terms for all the phages and proteins, together with two other variables: sex and age of the patients. The corrected AUC value was 0.83 with this model.

This model was probably more complex than necessary. For that reason, a variable selection was performed with the Akaike information criterion as the endpoint. The final model only retained 3 proteins (GRN, MST1 and SULF1), in addition to the age of the patients (Table 5). However, to prevent an overestimate of the predictive capacity of the model, estimated corrected AUC values were obtained by means of bootstrapping the entire variable selection process (i.e., the complete model with 8 variables was performed and the Akaike information criterion was used for each bootstrap sample). The corrected AUC was 0.84. Bootstrapping also provided information on selection process stability; most bootstrapping models contained four, five, six or seven variables. Some of the variables appeared in most of the models; the GRN phage in 976, protein SULF1 in 954, age in 952 and MST1 protein in 833.

This model was further used for predicting the probability of being CRC from a group of 57 sera that comprised various pathologies. A dot-plot (FIG. 6D) was generated, showing the individual probability for each subject. Great variability in probability was observed within each group, but the median was way below 0.5, indicating a low probability of having CRC.

TABLE 5
Final model with bootstrapping after the selection model

| | | Std | z | |
|---|---|---|---|---|
| | Estimate | Error. | value | Pr(>\|z\|) |
| (Intercept*) | −5.92318 | 2.15625 | −2.747 | 0.00601** |
| GRN-phage | −6.28345 | 2.91418 | −2.156 | 0.03107* |
| NHSL1- phage | 6.40976 | 2.8367 | 2.26 | 0.02385* |
| GTF2B- phage | −9.00788 | 2.63827 | −3.414 | 0.00064*** |
| SREBF2- | 10.31184 | 3.30888 | 3.116 | 0.00183** |
| phage | | | | |
| MST1- phage | 3.53576 | 1.32002 | 2.679 | 0.00739** |
| SULF1- phage | 7.26445 | 2.97056 | 2.445 | 0.01447* |
| Sex | −0.98593 | 0.65076 | −1.515 | 0.12976 |
| Age | 0.05674 | 0.0232 | 2.446 | 0.01444* |

*The intercept is the log value (p/(1 − p)), where p is the probability of being a tumor, when the value of the other variables in the model is 0. Estimate, estimated coefficient (slope); Std. Error, standard error of the specified variable; z value or Wald statistic, which is exactly equal to the estimated coefficient divided by its standard error; Pr(>|z|), p value of the Wald test for that specific coefficient comparing the z value to the normal standard;
*degree of significance.

The predictor was subsequently tested according to the stage of the patient, using the model with 6 markers (4 phages+2 proteins) plus the age of the patients. The AUC corrected using bootstrapping was 0.786 for stages A+B; 0.857 for stage C; and 0.849 for stage D. If the same test with CEA values is applied, the corrected AUC values were 0.742 for stages A+B, 0.770 for stage C and 0.973 for stage D. These results indicate clear superiority of the predictive model for the diagnosis of CRC in stages A, B and C, CEA being better for stage D, as was expected.

### Example 5

### Autoantibodies Against MST1 and NHSL1 are Associated with the Clinical Prognosis of CRC Patients

After having demonstrated that the predictive panel was capable of identifying patients with tumors in both early and late stages, the potential prognosis of autoantibodies was investigated by analyzing their association with absolute patient survival.

Survival was estimated by means of standard Kaplan-Meier method. The prognostic value of candidate antibodies was evaluated by means of stage-stratified Cox's proportional hazards models. The p values were obtained from the probability ratio test. Only the antibodies found as being significant for diagnosis were evaluated for prognosis in a step-by-step multivariate process.

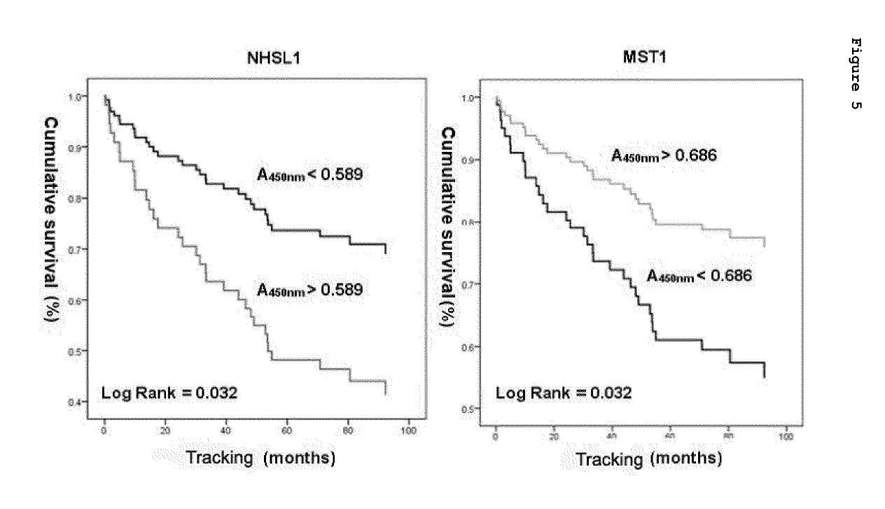

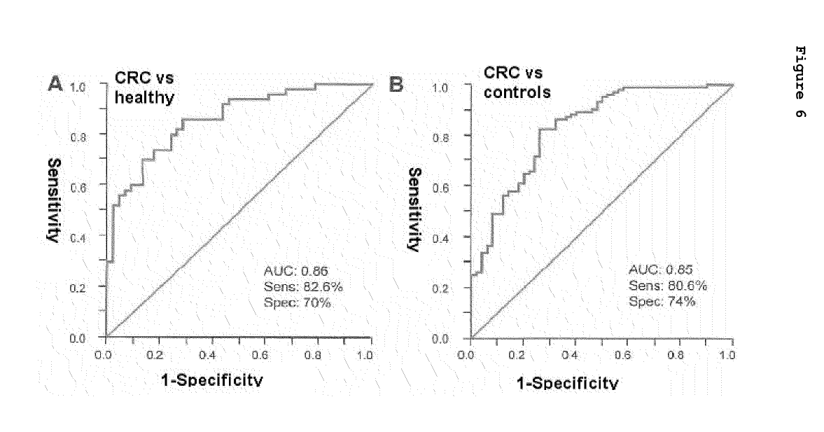

Patients with low levels of antibodies against MST1 showed a lower cumulative survival than those with high levels of autoantibodies (p=0.08). In contrast, patients with higher titers of antibodies against NHSL1 were associated with lower survival (p=0.06). The combination of both effects improves the statistical value of the prediction (0.032) (FIG. 5) and confirms the survival prediction. Collectively, these data indicate a correlation between the presence of antibodies against MST1 and NHSL1 with the clinical prognosis of colorectal cancer patients.

This prognostic value was most important for early stages of the disease with a risk index of 5.1; though it was not significant (p=0.12) because only 8 events were observed in the 47 patients in stages I-II. A similar effect was further observed in the analysis of disease-free survival when the patients were restricted to tumor-free surgical resection margins.

1. A method for detecting an autoantibody in a subject suspected of having colorectal cancer (CRC), comprising:
a) contacting a sample from said subject with an antibody capturing entity (ACE), wherein said ACE is selected from the group consisting of:
(i) an ACE comprising the amino acid sequence shown in SEQ ID NO: 4 or a variant thereof containing an epitope recognizable by an autoantibody;
(ii) an ACE comprising the amino acid sequence shown in SEQ ID NO: 2 or a variant thereof containing an epitope recognizable by an autoantibody;
(iii) an ACE comprising the amino acid sequence shown in SEQ ID NO: 6 or a variant thereof containing an epitope recognizable by an autoantibody;
(iv) an ACE comprising the amino acid sequence shown in SEQ ID NO: 3 or a variant thereof containing an epitope recognizable by an autoantibody, wherein said ACE is not MST1 protein;
(v) an ACE comprising the amino acid sequence shown in SEQ ID NO: 5 or a variant thereof containing an epitope recognizable by an autoantibody;
(vi) an ACE comprising the amino acid sequence shown in SEQ ID NO: 1 or a variant thereof containing an epitope recognizable by an autoantibody; and
(vii) any combination of said ACEs (i)-(vi); and
b) detecting the formation of an autoantibody-ACE complex,
wherein the detection of said autoantibody-ACE complex is indicative of the presence of said autoantibody in said subject.

2. The method according to claim 1, comprising the detection of an autoantibody against an ACE comprising the amino acid sequence shown in SEQ ID NO: 4 or a variant thereof containing an epitope recognizable by an autoantibody, and furthermore the detection of an autoantibody selected from the group consisting of:
(i) an autoantibody against an ACE comprising the amino acid sequence shown in SEQ ID NO: 2 or a variant thereof containing an epitope recognizable by an autoantibody;
(ii) an autoantibody against an ACE comprising the amino acid sequence shown in SEQ ID NO: 6 or a variant thereof containing an epitope recognizable by an autoantibody;
(iii) an autoantibody against an ACE comprising the amino acid sequence shown in SEQ ID NO: 3 or a variant thereof containing an epitope recognizable by an autoantibody;
(iv) an autoantibody against an ACE comprising the amino acid sequence shown in SEQ ID NO: 5 or a variant thereof containing an epitope recognizable by an autoantibody;
(v) an autoantibody against an ACE comprising the amino acid sequence shown in SEQ ID NO: 1 or a variant thereof containing an epitope recognizable by an autoantibody; and
(vi) any combination of autoantibodies (i) to (v).

3. The method according to claim 1, comprising the detection of an autoantibody against an ACE comprising the amino acid sequence shown in SEQ ID NO: 1 or a variant thereof containing an epitope recognizable by an autoantibody, and furthermore the detection of an autoantibody selected from the group consisting of:
(i) an autoantibody against an ACE comprising the amino acid sequence shown in SEQ ID NO: 4 or a variant thereof containing an epitope recognizable by an autoantibody;
(ii) an autoantibody against an ACE comprising the amino acid sequence shown in SEQ ID NO: 2 or a variant thereof containing an epitope recognizable by an autoantibody;
(iii) an autoantibody against an ACE comprising the amino acid sequence shown in SEQ ID NO: 6 or a variant thereof containing an epitope recognizable by an autoantibody;
(iv) an autoantibody against an ACE comprising the amino acid sequence shown in SEQ ID NO: 3 or a variant thereof containing an epitope recognizable by an autoantibody;
(v) an autoantibody against an ACE comprising the amino acid sequence shown in SEQ ID NO: 5 or a variant thereof containing an epitope recognizable by an autoantibody; and
(vi) any combination of autoantibodies (i) to (v).

4. (canceled)

5. The method according to claim 4, wherein said biological fluid comprises blood, plasma or blood serum.

6. (canceled)

7. The method according to claim 1, wherein said ACE is SULF1 or a variant or fragment thereof containing an epitope recognizable by an autoantibody.

8. The method according to claim 1, wherein said ACE is selected from the group consisting of:
(i) a phage comprising the amino acid sequence shown in SEQ ID NO: 4 or a variant thereof containing an epitope recognizable by an autoantibody, wherein said amino acid sequence is exposed on the phage surface;
(ii) a phage comprising the amino acid sequence shown in SEQ ID NO: 2 or a variant thereof containing an epitope recognizable by an autoantibody, wherein said amino acid sequence is exposed on the phage surface;
(iii) a phage comprising the amino acid sequence shown in SEQ ID NO: 6 or a variant thereof containing an epitope recognizable by an autoantibody, wherein said amino acid sequence is exposed on the phage surface;
(iv) a phage comprising the amino acid sequence shown in SEQ ID NO: 3 or a variant thereof containing an epitope recognizable by an autoantibody, wherein said amino acid sequence is exposed on the phage surface;
(v) a phage comprising the amino acid sequence shown in SEQ ID NO: 5 or a variant thereof containing an epitope recognizable by an autoantibody, wherein said amino acid sequence is exposed on the phage surface;
(vi) a phage comprising the amino acid sequence shown in SEQ ID NO: 1 or a variant thereof containing an epitope recognizable by an autoantibody, wherein said amino acid sequence is exposed on the phage surface; and
(vii) any of the combinations of (i) to (vi).

9. The method according to claim 1, wherein the detection of said autoantibodies is carried out by means of an immunoassay.

10. (canceled)

11. (canceled)

12. The method according to claim 1, further comprising correlating the formation of the autoantibody-ACE complex in the sample from the subject with a diagnosis of CRC.

13. The method according to claim 1, further comprising comparing the immunoreactivity of the sample with the immunoreactivity of a second sample from the same subject in a later time period.

14. The method according to claim 13, wherein said second sample from the subject has been obtained after said subject has been treated for CRC.

15. A method for detecting an autoantibody in a sample, comprising:
a) contacting said sample with an antibody capturing entity (ACE), wherein said ACE is selected from the group consisting of:
(i) an ACE comprising the amino acid sequence shown in SEQ ID NO: 4 or a variant thereof containing an epitope recognizable by an autoantibody;
(ii) an ACE comprising the amino acid sequence shown in SEQ ID NO: 2 or a variant thereof containing an epitope recognizable by an autoantibody;
(iii) an ACE comprising the amino acid sequence shown in SEQ ID NO: 6 or a variant thereof containing an epitope recognizable by an autoantibody;
(iv) an ACE comprising the amino acid sequence shown in SEQ ID NO: 3 or a variant thereof containing an epitope recognizable by an autoantibody, wherein said ACE is not MST1 protein;
(v) an ACE comprising the amino acid sequence shown in SEQ ID NO: 5 or a variant thereof containing an epitope recognizable by an autoantibody;
(vi) an ACE comprising the amino acid sequence shown in SEQ ID NO: 1 or a variant thereof containing an epitope recognizable by an autoantibody; and
(vii) any combination of said ACEs (i)-(vi); and
b) detecting the formation of an autoantibody-ACE complex,
wherein the detection of said autoantibody-ACE complex is indicative of the presence of said autoantibody in said sample.

16. The method according to claim 15, comprising the detection of an autoantibody against an ACE comprising the amino acid sequence shown in SEQ ID NO: 4 or a variant thereof containing an epitope recognizable by an autoantibody, and furthermore the detection of an autoantibody selected from the group consisting of:
(i) an autoantibody against an ACE comprising the amino acid sequence shown in SEQ ID NO: 2 or a variant thereof containing an epitope recognizable by an autoantibody;
(ii) an autoantibody against an ACE comprising the amino acid sequence shown in SEQ ID NO: 6 or a variant thereof containing an epitope recognizable by an autoantibody;
(iii) an autoantibody against an ACE comprising the amino acid sequence shown in SEQ ID NO: 3 or a variant thereof containing an epitope recognizable by an autoantibody;
(iv) an autoantibody against an ACE comprising the amino acid sequence shown in SEQ ID NO: 5 or a variant thereof containing an epitope recognizable by an autoantibody;
(v) an autoantibody against an ACE comprising the amino acid sequence shown in SEQ ID NO: 1 or a variant thereof containing an epitope recognizable by an autoantibody; and
(vi) any combination of autoantibodies (i) to (v).

17. The method according to claim 15, comprising the detection of an autoantibody against an ACE comprising the amino acid sequence shown in SEQ ID NO: 1 or a variant thereof containing an epitope recognizable by an autoantibody, and furthermore the detection of an autoantibody selected from the group consisting of:
(i) an autoantibody against an ACE comprising the amino acid sequence shown in SEQ ID NO: 4 or a variant thereof containing an epitope recognizable by an autoantibody;
(ii) an autoantibody against an ACE comprising the amino acid sequence shown in SEQ ID NO: 2 or a variant thereof containing an epitope recognizable by an autoantibody;
(iii) an autoantibody against an ACE comprising the amino acid sequence shown in SEQ ID NO: 6 or a variant thereof containing an epitope recognizable by an autoantibody;
(iv) an autoantibody against an ACE comprising the amino acid sequence shown in SEQ ID NO: 3 or a variant thereof containing an epitope recognizable by an autoantibody;
(v) an autoantibody against an ACE comprising the amino acid sequence shown in SEQ ID NO: 5 or a variant thereof containing an epitope recognizable by an autoantibody; and
(vi) any combination of autoantibodies (i) to (v).

18. An antibody capturing entity (ACE) selected from the group consisting of:
(i) an ACE comprising the amino acid sequence shown in SEQ ID NO: 4 or a variant thereof containing an epitope recognizable by an autoantibody, wherein said ACE is not SULF1 protein;
(ii) an ACE comprising the amino acid sequence shown in SEQ ID NO: 2 or a variant thereof containing an epitope recognizable by an autoantibody, wherein said ACE is not GRN protein;
(iii) an ACE comprising the amino acid sequence shown in SEQ ID NO: 6 or a variant thereof containing an epitope recognizable by an autoantibody, wherein said ACE is not GTF2i protein;
(iv) an ACE comprising the amino acid sequence shown in SEQ ID NO: 3 or a variant thereof containing an epitope recognizable by an autoantibody, wherein said ACE is not MST1 protein;
(v) an ACE comprising the amino acid sequence shown in SEQ ID NO: 5 or a variant thereof containing an epitope recognizable by an autoantibody, wherein said ACE is not SREBF2 protein;
(vi) an ACE comprising the amino acid sequence shown in SEQ ID NO: 1 or a variant thereof containing an epitope recognizable by an autoantibody, wherein said ACE is not NHSL1 protein; and
(vii) any combination of ACEs (i) to (vi).

19. (canceled)

20. The antibody capturing entity (ACE) according to claim 18, wherein said ACE is selected from the group consisting of:
(i) a phage comprising the amino acid sequence shown in SEQ ID NO: 1 or a variant thereof containing an epitope recognizable by an autoantibody, wherein said amino acid sequence is exposed on the phage surface;
(ii) a phage comprising the amino acid sequence shown in SEQ ID NO: 2 or a variant thereof containing an epitope recognizable by an autoantibody, wherein said amino acid sequence is exposed on the phage surface;
(iii) a phage comprising the amino acid sequence shown in SEQ ID NO: 3 or a variant thereof containing an epitope recognizable by an autoantibody, wherein said amino acid sequence is exposed on the phage surface;
(iv) a phage comprising the amino acid sequence shown in SEQ ID NO: 4 or a variant thereof containing an epitope recognizable by an autoantibody, wherein said amino acid sequence is exposed on the phage surface;
(v) a phage comprising the amino acid sequence shown in SEQ ID NO: 5 or a variant thereof containing an epitope recognizable by an autoantibody, wherein said amino acid sequence is exposed on the phage surface;
(vi) a phage comprising the amino acid sequence shown in SEQ ID NO: 6 or a variant thereof containing an epitope recognizable by an autoantibody, wherein said amino acid sequence is exposed on the phage surface; and
(vii) any of the combinations of (i) to (vi).

21. A composition comprising an antibody capturing entity (ACE) according to claim 18.

22. The composition according to claim 21, comprising at least one ACE according to claim 18, and at least one protein selected from the group consisting of SULF1 protein or a variant thereof, MST1 protein or a variant thereof, and their combinations.

23. The composition according to claim 22, comprising:
a) an ACE selected from the group consisting of:
(i) an ACE comprising the amino acid sequence shown in SEQ ID NO: 2 or a variant thereof containing an epitope recognizable by an autoantibody,
(ii) an ACE comprising the amino acid sequence shown in SEQ ID NO: 6 or a variant thereof containing an epitope recognizable by an autoantibody;
(iii) an ACE comprising the amino acid sequence shown in SEQ ID NO: 5 or a variant thereof containing an epitope recognizable by an autoantibody;
(iv) an ACE comprising the amino acid sequence shown in SEQ ID NO: 1 or a variant thereof containing an epitope recognizable by an autoantibody; and
(v) any combination of ACEs (i) to (iv); and
b) a protein selected from the group consisting of SULF1 protein or a variant thereof, MST1 protein or a variant thereof, and their combinations.

24. A composition comprising SULF1 protein or a variant thereof and MST1 protein or a variant thereof and an antibody capturing entity (ACE) according to claim 18.

25. The composition according to claim 24, comprising:
a) SULF1 protein or a variant thereof;
b) MST1 protein or a variant thereof; and
c) an ACE selected from the group consisting of:
(i) an ACE comprising the amino acid sequence shown in SEQ ID NO: 2 or a variant thereof containing an epitope recognizable by an autoantibody,
(ii) an ACE comprising the amino acid sequence shown in SEQ ID NO: 6 or a variant thereof containing an epitope recognizable by an autoantibody;
(iii) an ACE comprising the amino acid sequence shown in SEQ ID NO: 5 or a variant thereof containing an epitope recognizable by an autoantibody;
(iv) an ACE comprising the amino acid sequence shown in SEQ ID NO: 1 or a variant thereof containing an epitope recognizable by an autoantibody; and
(v) any combination of ACEs (i) to (iv).

26. (canceled)

27. A kit comprising a composition according to claim 21.

28. (canceled)

29. (canceled)

30. (canceled)
